(19) United States
(12) Patent Application Publication
(10) Pub. No.: US 2004/0077650 A1
(43) Pub. Date: Apr. 22, 2004

(54) Cannabinoid receptor ligands and uses thereof

(75) Inventor: Robert L. Dow, Waterford, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/679,878

(22) Filed: Oct. 6, 2003

Related U.S. Application Data
(60) Provisional application No. 60/419,621, filed on Oct. 18, 2002.

(51) Int. Cl. A61K 31/53; A61K 31/506; A61K 31/4196; C 07D 4 3/04

(52) U.S. Cl. 514/242; 514/256; 514/383; 514/397; 544/184; 544/333; 548/266.2

(57) ABSTRACT

Compounds of Formula (I) that act as cannabinoid receptor ligands and their uses in the treatment of diseases linked to the modulation of the cannabinoid receptors in animals are described herein.

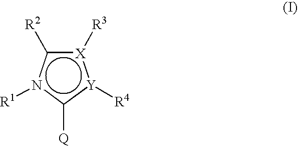

This application claims the benefit of U.S. Provisional Application No. 60/419,621 filed on Oct. 18, 2002 and incorporated herein by reference in its entirety.

### FIELD OF THE INVENTION

The present invention relates to bi-heteroaryl compounds as cannabinoid receptor ligands, in particular CB1 receptor antagonists or inverse agonists, and uses thereof for treating diseases, conditions and/or disorders modulated by cannabinoid receptor antagonists.

### BACKGROUND

Obesity is a major public health concern because of its increasing prevalence and associated health risks. Obesity and overweight are generally defined by body mass index (BMI), which is correlated with total body fat and estimates the relative risk of disease. BMI is calculated by weight in kilograms divided by height in meters squared (kg/m²). Overweight is typically defined as a BMI of 25-29.9 kg/m², and obesity is typically defined as a BMI of 30 kg/m². See, e.g., National Heart, Lung, and Blood Institute, Clinical Guidelines on the Identification, Evaluation, and Treatment of Overweight and Obesity in Adults, The Evidence Report, Washington, DC: U.S. Department of Health and Human Services, NIH publication no. 98-4083 (1998).

The increase in obesity is of concern because of the excessive health risks associated with obesity, including coronary heart disease, strokes, hypertension, type 2 diabetes mellitus, dyslipidemia, sleep apnea, osteoarthritis, gall bladder disease, depression, and certain forms of cancer (e.g., endometrial, breast, prostate, and colon). The negative health consequences of obesity make it the second leading cause of preventable death in the United States and impart a significant economic and psychosocial effect on society. See, McGinnis M, Foege W H., “Actual Causes of Death in the United States,” JAMA, 270, 2207-12 (1993).

Obesity is now recognized as a chronic disease that requires treatment to reduce its associated health risks. Although weight loss is an important treatment outcome, one of the main goals of obesity management is to improve cardiovascular and metabolic values to reduce obesity-related morbidity and mortality. It has been shown that 5-10% loss of body weight can substantially improve metabolic values, such as blood glucose, blood pressure, and lipid concentrations. Hence, it is believed that a 5-10% intentional reduction in body weight may reduce morbidity and mortality.

Currently available prescription drugs for managing obesity generally reduce weight by inducing satiety or decreasing dietary fat absorption. Satiety is achieved by increasing synaptic levels of norepinephrine, serotonin, or both. For example, stimulation of serotonin receptor subtypes 1B, 1D, and 2C and 1- and 2-adrenergic receptors decreases food intake by regulating satiety. See, Bray GA, “The New Era of Drug Treatment. Pharmacologic Treatment of Obesity: Symposium Overview,” Obes Res., 3(suppl 4), 415s-7s (1995). Adrenergic agents (e.g., diethylpropion, benzphetamine, phendimetrazine, mazindol, and phentermine) act by modulating central norepinephrine and dopamine receptors through the promotion of catecholamine release. Older adrenergic weight-loss drugs (e.g., amphetamine, methamphetamine, and phenmetrazine), which strongly engage in dopamine pathways, are no longer recommended because of the risk of their abuse. Fenfluramine and dexfenfluramine, both serotonergic agents used to regulate appetite, are no longer available for use.

More recently, CB1 cannabinoid receptor antagonists/inverse agonists have been suggested as potential appetite suppressants. See, e.g., Arnone, M., et al., “Selective Inhibition of Sucrose and Ethanol Intake by SR141716, an Antagonist of Central Cannabinoid (CB1) Receptors,” Psychopharmacol, 132, 104-106 (1997); Colombo, G., et al., “Appetite Suppression and Weight Loss after the Cannabinoid Antagonist SR141716,” Life Sci., 63, PL113-PL117 (1998); Simiand, J., et al., “SR141716, a CB1 Cannabinoid Receptor Antagonist, Selectively Reduces Sweet Food Intake in Marmose,” Behav. Pharmacol., 9, 179-181 (1998); and Chaperon, F., et al., “Involvement of Central Cannabinoid (CB1) Receptors in the Establishment of Place Conditioning in Rats,” Psychopharmacology, 135, 324-332 (1998). For a review of cannabinoid CB1 and CB2 receptor modulators, see Pertwee, R. G., “Cannabinoid Receptor Ligands: Clinical and Neuropharmacological Considerations, Relevant to Future Drug Discovery and Development,” Exp. Opin. Invest. Drugs, 9(7), 1553-1571 (2000).

Although investigations are on-going, there still exists a need for a more effective and safe therapeutic treatment for reducing or preventing weight-gain.

In addition to obesity, there also exists an unmet need for treatment of alcohol abuse. Alcoholism affects approximately 10.9 million men and 4.4 million women in the United States. Approximately 100,000 deaths per year have been attributed to alcohol abuse or dependence. Health risks associated with alcoholism include impaired motor control and decision making, cancer, liver disease, birth defects, heart disease, drug/drug interactions, pancreatitis and interpersonal problems. Studies have suggested that endogenous cannabinoid tone plays a critical role in the control of ethanol intake. The endogenous CB1 receptor antagonist SR-141716A has been shown to block voluntary ethanol intake in rats and mice. See, Arnone, M., et al., “Selective Inhibition of Sucrose and Ethanol Intake by SR141716, an Antagonist of Central Cannabinoid (CB1) Receptors,” Psychopharmacol, 132,104-106 (1997). For a review, see Hungund, B. L and B. S. Basavarajappa, “Are Anadamide and Cannabinoid Receptors involved in Ethanol Tolerance? A Review of the Evidence,” Alcohol & Alcoholism. 35(2) 126-133, 2000.

Current treatments for alcohol abuse or dependence generally suffer from non-compliance or potential hepatotoxicity; therefore, there is a high unmet need for more effective treatment of alcohol abuse/dependence.

### SUMMARY

The present invention provides compounds of Formula (I) that act as cannabinoid receptor ligands (preferably, CB1 receptor antagonists or inverse agonists).

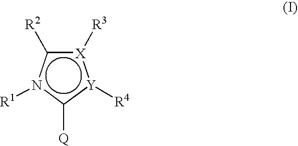

wherein

X is carbon and Y is nitrogen, or X is nitrogen and Y is carbon;

R¹ is a lone pair of electrons, hydrogen, (C₁-C₆)alkyl, or (C₃-C₆)cycloalkyl;

R² is hydrogen, (C₁-C₆)alkyl, or (C₃-C₆)cycloalkyl;

R³ is hydrogen or a chemical moiety selected from the group consisting of (C₁-C₆)alkyl, 2- to 8-membered carbocyclic ring, 5- to 6-membered heterocyclic ring, aryl, 5- to 9-membered heteroaryl, (C₁-C₆)alkylaryl (e.g., tolyl, etc.), (C₁-C₆)alkylheteroaryl, aryl(C₁-C₆)alkyl (e.g., benzyl, 1-methyl-1-phenyl-ethyl, α-phenethyl, and the like), aryloxy(C₁-C₆)alkyl when X is carbon or nitrogen, where the chemical moiety is optionally substituted, or

R³ is a lone pair of electrons when X is nitrogen;

R⁴ is hydrogen or a chemical moiety selected from the group consisting of (C₁-C₆)alkyl, aryl, and aryl(C₁-C₆)alkyl when Y is carbon or nitrogen, where the chemical moiety is optionally substituted, or

R⁴ is a lone pair of electrons when Y is nitrogen; and

Q is a group selected from

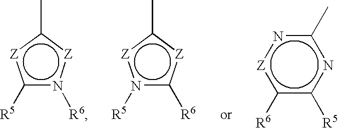

where Z in each occurrence is independently nitrogen or CR⁷, R⁵ is an optionally substituted aryl, or an optionally substituted heteroaryl (preferably, the aryl and heteroaryl groups are each independently substituted with one to three substituents selected from halo, (C₁-C₄)alkoxy, (C₁-C₄)alkyl, halo-substituted(C₁-C₄)alkyl (e.g., CH₂F, CHF₂ and CF₃) and cyano, more preferably, R⁵ is 2,4-dihalophenyl or 2-halophenyl, most preferably, 2,4-dichlorophenyl, 2-chlorophenyl, or 2-fluorophenyl), R⁶ is an optionally substituted aryl, or an optionally substituted heteroaryl (preferably, the aryl and heteroaryl substituents are selected from the group consisting of halo, (C₁-C₄)alkoxy, (C₁-C₄)alkyl, halo-substituted(C₁-C₄)alkyl (e.g., CH₂F, CHF₂ and CF₃) and cyano, more preferably, R⁶ is p-halophenyl or 2-(C₁-C₆)alkoxypyridin-5-yl, most preferably p-chlorophenyl, p-fluorophenyl, or 2-methoxypyridin-5-yl), and R⁷ is hydrogen, halo, cyano, or (C₁-C₆)alkyl; a pharmaceutically acceptable salt thereof, a prodrug of the compound or salt, or a solvate or hydrate of the compound, salt or prodrug.

In a preferred embodiment, a compound having Formula (IA) or Formula (1B) below is provided.

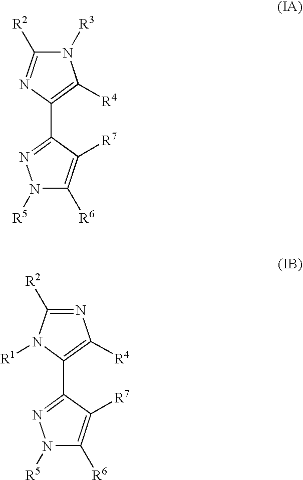

wherein R¹, R², R³, R⁴, R⁵, R⁶ and R⁷ have the same meanings as defined above; a pharmaceutically acceptable salt thereof, a prodrug of the compound or the salt, or a solvate or hydrate of the compound, the salt or the prodrug. Even more preferred are compounds of Formula (IA).

In another preferred embodiment, a compound of Formula (IC) or Formula (1D) is provided.

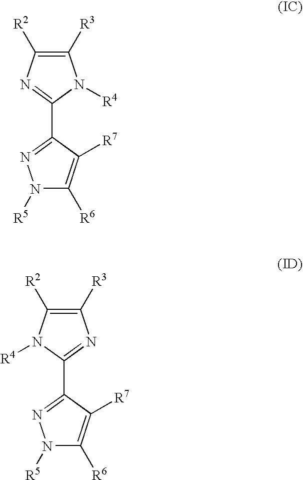

wherein R², R³, R⁴, R⁵, R⁶ and R⁷ have the same meanings as defined above; a pharmaceutically acceptable salt thereof, a prodrug of the compound or the salt, or a solvate or hydrate of the compound, the salt or the prodrug.

Preferred compounds of the present invention include: 5-(4-chloro-phenyl-3-(5-cyclohexyl-1H-imidazol-2-yl)-1-(2,4-dichloro-phenyl)-4-methyl-1H-pyrazole; 5-(4-chloro-phenyl)-3-(2-cyclohexyl-3H-imidazol-4-yl)-1-(2,4-dichloro-phenyl)-4-methyl-1H-pyrazole; 5-(4-chloro-phenyl)-1-(2,4-dichloro-phenyl)-4-methyl-3-[1-(1-methyl-1-phenyl-ethyl)-1H-imidazol-4-yl]-1H-pyrazole; 5-(4-chloro-phenyl)-1-(2-chloro-phenyl)-4-methyl-3-[1-(1-phenyl-ethyl)-1H-imidazol-4-yl]-1H-pyrazole; 5-(4-chloro-phenyl)-1-(2-fluoro-phenyl)-4-methyl-3-[1-(1-methyl-1-phenyl-ethyl)-1H-imidazol-4-yl]-1H-pyrazole; 5-(4-chloro-phenyl)-1-(2-chloro-phenyl)-3-[1 -(2,2-dimethyl-tetrahydro-pyran-4-yl)-1H-imidazol-4-yl]-4-methyl-1H-pyrazole: 5-{2-(2,4-dichloro-phenyl)-4-methyl-5-[1-(1-methyl-1-phenyl-ethyl)-1H-imidazol-4-yl]-2H-pyrazol-3-yl}-2-methoxy-pyridine; and 1-(2-chloro-phenyl)-5-(4-chloro-phenyl)-4-methyl-3-[1-(1-methyl-1-phenyl-ethyl)-1H-imidazol-4-yl]-1H-pyrazole; a pharmaceutically acceptable salt thereof or a solvate or hydrate of the compound or the salt.

Some of the compounds described herein contain at least one chiral center; consequently, those skilled in the art will appreciate that all stereoisomers (e.g., enantiomers and diasteroisomers) of the compounds illustrated and discussed herein are within the scope of the present invention. In addition, tautomeric forms of the compounds are also within the scope of the present invention.

In another embodiment of the present invention, a pharmaceutical composition is provided that comprises (1) a compound of the present invention, and (2) a pharmaceutically acceptable excipient, diluent, or carrier.

In yet another embodiment of the present invention, a method for treating a disease, condition or disorder modulated by a cannabinoid receptor (preferably, the CB1 receptor) antagonist in animals that includes the step of administering to an animal in need of such treatment a therapeutically effective amount of a compound of the present invention (or a pharmaceutical composition thereof).

Diseases, conditions, and/or disorders modulated by cannabinoid receptor antagonists include eating disorders (e.g., binge eating disorder, anorexia, and bulimia), weight loss or control (e.g., reduction in calorie or food intake, and/or appetite suppression), obesity, depression, atypical depression, bipolar disorders, psychoses, schizophrenia, behavioral addictions, suppression of reward-related behaviors (e.g., conditioned place avoidance, such as suppression of cocaine- and morphine-induced conditioned place preference), substance abuse, addictive disorders, impulsivity, alcoholism (e.g., alcohol abuse, addiction and/or dependence including treatment for abstinence, craving reduction and relapse prevention of alcohol intake), tobacco abuse (e.g., smoking addiction, cessation and/or dependence including treatment for craving reduction and relapse prevention of tobacco smoking), dementia (including memory loss, Alzheimer's disease, dementia of aging, vascular dementia, mild cognitive impairment, age-related cognitive decline, and mild neurocognitive disorder), sexual dysfunction in males (e.g., erectile difficulty), seizure disorders, epilepsy, gastrointestinal disorders (e.g., dysfunction of gastrointestinal motility or intestinal propulsion), attention deficit disorder (ADD including attention deficit hyperactivity disorder (ADHD)), Parkinson's disease, and type II diabetes. In a preferred embodiment, the method is used in the treatment of obesity, attention deficit disorder, alcoholism, and/or tobacco abuse.

Compounds of the present invention may be administered in combination with other pharmaceutical agents. Preferred pharmaceutical agents include nicotine receptor partial agonists, opioid antagonists (e.g., naltrexone (including naltrexone depot), antabuse, and nalmefene), dopaminergic agents (e.g., apomorphine), ADD/ADHD agents (e.g., methylphenidate hydrochloride (e.g., Ritalin™ and Concerta™), atomoxetine (e.g., Strattera™), and amphetamines (e.g., Adderall™)) and anti-obesity agents, such as apo-B/MTP inhibitors, 11β-hydroxy steroid dehydrogenase-1 (11β-HSD type 1) inhibitors, peptide YY₃₋₃₆ or analogs thereof, MCR-4 agonists, CCK-A agonists, monoamine reuptake inhibitors, sympathomimetic agents, β₃ adrenergic receptor agonists, dopamine receptor agonists, melanocyte-stimulating hormone receptor analogs, 5-HT2c receptor agonists, melanin concentrating hormone receptor antagonists, leptin, leptin analogs, leptin receptor agonists, galanin receptor antagonists, lipase inhibitors, bombesin receptor agonists, neuropeptide-Y receptor antagonists, thyromimetic agents, dehydroepiandrosterone or analogs thereof, glucocorticoid receptor antagonists, orexin receptor antagonists, glucagon-like peptide-1 receptor agonists, ciliary neurotrophic factors, human agouti-related protein antagonists, ghrelin receptor antagonists, histamine 3 receptor antagonists or inverse agonists, and neuromedin U receptor agonists, and the like.

The combination therapy may be administered as (a) a single pharmaceutical composition which comprises a compound of the present invention, at least one additional pharmaceutical agent described above and a pharmaceutically acceptable excipient, diluent, or carrier; or (b) two separate pharmaceutical compositions comprising (i) a first composition comprising a compound of Formula (I) and a pharmaceutically acceptable excipient, diluent, or carrier, and (ii) a second composition comprising at least one additional pharmaceutical agent described above and a pharmaceutically acceptable excipient, diluent, or carrier. The pharmaceutical compositions may be administered simultaneously or sequentially and in any order.

In yet another aspect of the present invention, a pharmaceutical kit is provided for use by a consumer to treat diseases, conditions or disorders modulated by cannabinoid receptor antagonists in an animal. The kit comprises a) a suitable dosage form comprising a compound of the present invention; and b) instructions describing a method of using the dosage form to treat diseases linked to the modulation of the cannabinoid receptor (preferably, the CB1 receptor).

In yet another embodiment of the present invention is a pharmaceutical kit comprising: a) a first dosage form comprising (i) a compound of the present invention and (ii) a pharmaceutically acceptable carrier, excipient or diluent; b) a second dosage form comprising (i) an additional pharmaceutical agent described above, and (ii) a pharmaceutically acceptable carrier, excipient or diluent; and c) a container.

### Definitions

As used herein, the term “alkyl” refers to a hydrocarbon radical of the general formula CₙH₂ₙ₊₁. The alkane radical may be straight or branched. For example, the term “(C₁-C₆)alkyl” refers to a monovalent, straight, or branched aliphatic group containing 1 to 6 carbon atoms (e.g., methyl, ethyl, n-propyl, i-propyl, n-butyl, i-butyl, s-butyl, t-butyl, n-pentyl, 1-methylbutyl, 2-methylbutyl, 3-methylbutyl, neopentyl, 3,3-dimethylpropyl, hexyl, 2-methylpentyl, and the like). Unless specified otherwise, the alkane radical may be optionally substituted with one or more substituents (generally, one to three substituents except in the case of halogen substituents such as perchloro or perfluoroalkyls) selected from the group of substituents listed below in the definition for “substituted.” For example, “halo-substituted alkyl” refers to an alkyl group substituted with one or more halogen atoms (e.g., fluoromethyl, difluoromethyl, trifluoromethyl, perfluoroethyl, and the like). Similarly, the alkyl portion of an alkoxy, alkylamino, dialkylamino, alkylaryl, alkylheteroaryl, and alkylthio group has the same definition as above.

The terms “partially or fully saturated carbocyclic ring” (also referred to as “partially or fully saturated cycloalkyl”) refers to nonaromatic rings that are either partially or fully hydrogenated and may exist as a single ring, bicyclic ring or a spiro-fused ring. For example, partially or fully saturated carbocyclic rings (or cycloalkyl) include groups such as cyclopropyl, cyclopropenyl, cyclobutyl, cyclobutenyl, cyclopentyl, cyclpentenyl, cyclopentadienyl, cyclohexyl, cyclohexenyl, cyclohexadienyl, norbornyl (bicyclo[2.2.1]heptyl), norbornenyl, bicyclo[2.2.2]octyl, and the like. Generally, the carbocyclic ring is a 3 to 8 membered ring. In addition, the partially saturated or fully saturated cycloalkyl may be optionally substituted with one of more substituents (typically, one to three substituents) selected from the group of substituents listed below in the definition for “substituted.” A substituted carbocyclic or heterocyclic ring includes groups wherein the carbocyclic ring is fused to a phenyl ring (e.g., indanyl, etc.) or a heteroaryl ring. The carbocyclic group may be attached to the chemical entity or moiety by any one of the carbon atoms within the carbocyclic ring system.

The term “partially saturated or fully saturated heterocyclic ring” (also referred to as “heterocycle”) refers to nonaromatic rings that are either partially or fully hydrogenated and may exist as a single ring, bicyclic ring or a spiro-fused ring. Partially saturated or fully saturated heterocyclic rings include groups such as epoxy, aziridinyl, tetrahydrofuranyl, dihydrofuranyl, dihydropyridinyl, pyrrolidinyl, N-methylpyrrolidinyl, imidazolidinyl, imidazolinyl, piperidinyl, piperazinyl, pyrazolidinyl, 2H-pyranyl, 4H-pyranyl, 2H-chromenyl, oxazinyl, morpholino, thiomorpholino, tetrahydrothienyl, tetrahydrothienyl 1,1-dioxide, and the like. Generally, the heterocycle is 3 to 8 membered ring containing 1 to 3 heteroatoms selected from oxygen, sulfur and nitrogen. Unless specified otherwise, the partially saturated or fully saturated heterocyclic groups may be optionally substituted with one of more substituents (typically, one to three substituents) selected from the group of substituents listed below in the definition for “substituted.” A substituted heterocyclic ring includes groups wherein the heterocyclic ring is fused to a phenyl ring (e.g., 2,3-dihydrobenzofuranyl, 2,3-dihydroindolyl, 2,3-dihydrobenzothiophenyl, 2,3-dihydrobenzothiazolyl, etc.) or a heteroaryl ring. The heterocyclic group may be attached to the chemical entity or moiety by any one of the atoms within the heterocyclic ring system.

The term “aryl” or “aromatic carbocyclic ring” refers to aromatic moieties having single (e.g., phenyl) or fused ring system (e.g., naphthalene, anthracene, phenanthrene, etc.). Unless indicated otherwise, the aryl groups may be optionally substituted with one or more substituents (preferably no more than three substituents) selected from the group of substituents listed below in the definition for “substituted.” Substituted aryl groups include a chain of aromatic moieties (e.g., biphenyl, terphenyl, phenylnaphthalyl, etc.) The aryl group may be attached to the chemical entity or moiety by any one of the carbon atoms within the aromatic ring system. Preferred aryl substituents are halogens (F, Cl, Br or I, preferably F or Cl), (C₁-C₄)alkoxy, (C₁-C₄)alkyl, halo-substituted(C₁-C₄)alkyl (e.g., CH₂F, CHF₂ and CF₃) and cyano. Similarly, the aryl portion (i.e., aromatic moiety) of an aroyl or aroyloxy (i.e., (aryl)-C(O)—O—) has the same definition as above.

The term “heteroaryl” or “heteroaromatic ring” refers to aromatic moieties containing at least one heteratom (e.g., oxygen, sulfur, nitrogen or combinations thereof) within the aromatic ring system (e.g., pyrrolyl, pyridyl, pyrazolyl, indolyl, indazolyl, thienyl, furanyl, benzofuranyl, oxazolyl, imidazolyl, tetrazolyl, triazinyl, pyrimidyl, pyrazinyl, thiazolyl, purinyl, benzimidazolyl, quinolinyl, isoquinolinyl, benzothiophenyl, benzoxazolyl, etc.). The heteroaromatic moiety may consist of a single or fused ring system. A typical single heteroaryl ring is a 5- to 6-membered ring containing one to three heteroatoms selected from oxygen, sulfur and nitrogen and a typical fused heteroaryl ring system is a 9- to 10-membered ring system containing one to four heteroatoms selected from oxygen, sulfur and nitrogen. Unless specified otherwise, the heteroaryl groups may be optionally substituted with one or more substituents (preferably no more than three substituents) selected from the group of substituents listed below in the definition for “substituted.” The heteroaryl group may be attached to the chemical entity or moiety by any one of the atoms within the aromatic ring system (e.g., imidazol-1-yl, imidazol-2-yl, imidazol-4-yl, imidazol-5-yl, pyrid-2-yl, pyrid-3-yl, pyrid-4-yl, pyrid-5-yl, or pyrid-6-yl). Similarly, the heteroaryl portion (i.e., heteroaromatic moiety) of a heteroarylalkyl has the same definition as above.

The term “halo” or “halogen” refers to chlorine, bromine, fluorine or iodine.

The term “substituted” specifically envisions and allows for substitutions that are common in the art. However, it is generally understood by those skilled in the art that the substituents should be selected so as to not adversely affect the pharmacological characteristics of the compound or adversely interfere with the use of the medicament. Those skilled in the art will also appreciate that some substitutions may be inherently unstable and therefore do not form a part of this invention. Suitable substituents for any of the groups defined above include (C₁-C₆)alkyl, (C₃-C₇)cycloalkyl, (C₂-C₆)alkenyl, aryl, heteroaryl, 3- to 6-membered heterocycle, halo (e.g., chloro, bromo, iodo and fluoro), cyano, hydroxy, (C₁-C₆)alkoxy, aryloxy, sulfhydryl (mercapto), (C₁-C₆)alkylthio, arylthio, amino, mono- or di-(C₁-C₆)alkyl amino, quaternary ammonium salts, amino(C₁-C₆)alkoxy, aminocarboxylate (i.e., —NH—C(O)—O—(C₁-C₆)alkyl), hydroxy(C₁-C₆)alkylamino, amino(C₁-C₆)alkylthio, cyanoamino, nitro, (C₁-C₆)carbamyl, keto (oxy), (C₁-C₆)carbonyl, (C₁-C₆)carboxy, glycolyl, glycyl, hydrazino, guanyl, sulfamyl, sulfonyl, sulfinyl, thio(C₁-C₆)carbonyl, thio(C₁-C₆)carboxy, and combinations thereof. In the case of substituted combinations, such as “substituted aryl(C₁-C₆)alkyl”, either the aryl or the alkyl group may be substituted, or both the aryl and the alkyl groups may be substituted with one or more substituents (typically, one to three substituents except in the case of perhalo substitutions). An aryl substituted carbocyclic or heterocyclic group may be a fused ring (e.g., indanyl, dihydrobenzofuranyl, dihydroindolyl, etc.).

The term “solvate” refers to a molecular complex of a compound represented by Formula (I) or (IA) (including prodrugs and pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The term “protecting group” or “Pg” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBz) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include —CH₂CH₂SO₂Ph, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see T. W. Greene, Protective Groups in Organic Synthesis, John Wiley & Sons, New York, 1991.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “animal” refers to humans (male and female), companion animals (e.g., dogs, cats and horses), food-source animals, zoo animals, marine animals, birds and other similar animal species. “Edible animals” refers to food-source animals such as cows, pigs, sheep and poultry.

The phrase “pharmaceutically acceptable” indicates that the substance or composition must be compatible chemically and/or toxicologically, with the other ingredients comprising a formulation, and/or the mammal being treated therewith.

The terms “treating”, “treat”, or “treatment” embrace both preventative, i.e., prophylactic, and palliative treatment.

The phrase “modulated by a cannabinoid receptor” refers to the activation or deactivation of a cannabinoid receptor. For example, the ligand may act as an agonist, partial agonist, inverse agonist, antagonist, or partial antagonist.

The term “antagonist” refers to both full and partial antagonists as well as inverse agonists.

The term “compounds of the present invention” (unless specifically identified otherwise) refer to compounds of Formula (I), (IA), (IB), (IC) or (ID), prodrugs thereof, pharmaceutically acceptable salts of the compounds, and/or prodrugs, and hydrates or solvates of the compounds, salts, and/or prodrugs, as well as, all stereoisomers (including diastereoisomers and enantiomers), tautomers and isotopically labeled compounds.

### DETAILED DESCRIPTION

The present invention provides compounds and pharmaceutical formulations thereof that are useful in the treatment diseases, conditions and/or disorders modulated by cannabinoid receptor antagonists.

Compounds of the present invention may be synthesized by synthetic routes that include processes analogous to those well-known in the chemical arts, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In the preparation of compounds of the present invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (BOC), benzyloxycarbonyl (CBz) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

Scheme I illustrates a method for preparing 1,4-disubstituted and 1,4,5-trisubstituted imidazoles (e.g., compounds of Formula (I), where R³, or R³ and R⁴ are other than hydrogen and X is nitrogen). The synthetic route outlined in Scheme I below is based on the synthetic procedures described by Sisko, J. et al., in J. Org. Chem., 65, 1516 (2000) and Org. Syn. 77, 198 (1999).

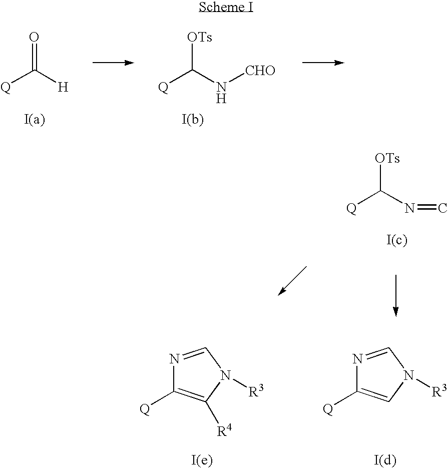

Aldehyde I(a) may be prepared from well-known procedures in the literature. For example, aldehydes I(a) where Q is a substituted or unsubstituted 1,5-diphenylpyrazole derivative can be prepared from its corresponding carboxylic acid or ester by reducing the ester with lithium aluminum hydride followed by oxidation with a suitable oxidizing agent (e.g. CrO₃ in pyridine) to produce the aldehyde I(a). General procedures for preparing the carboxylic acid, ester and/or aldehyde are described in U.S. Pat. Nos. 4,944,790, 5,051,518, 5,134,142, and 5,624,941, all of which are incorporated herein by reference, and Bischler, Chemische Berichte, 26, 1881-1890 (1893). Other 1,5-disubstituted aryl and heteroaryl pyrazole aldehyde derivatives may be prepared using analogous procedures. The corresponding pyrimidine-based aldehydes can be prepared using procedures outlined in: WO9202513. The corresponding imidazole intermediates can be prepared using procedures outlined in: U.S. Pat. No. 5,616,601 (incorporated herein by reference) or C. Gonczi and H. Vander Plas, J. Org. Chem., 46(3), 608-610 (1981). The corresponding triazole intermediates can be prepared using procedures described in: Liebigs Ann. Chem. 48-65 (1984).

Aldehyde (I(a)) is heated with formamide and p-toluenesulfinic acid in the presence of trimethylsilylchloride (TMSCI) in an aprotic solvent (e.g., touene/acetonitrile) to produce intermediate I(b). The tosylmethyl isocyanide I(c) is then prepared by reacting intermediate I(b) with phosphorousoxy-chloride (POCl₃) in the presence of an amine (e.g., triethylamine) in an aprotic solvent (e.g., THF).

In the final step, the desired polysubstitued imidazole I(d) or I(e) is prepared in a single pot from the tosylmethyl isocyanide I(c) and the appropriate imine generated in situ. For example, reaction of tosylmethyl isocyanide I(c) with glyoxylic acid and the appropriate amine (i.e., R³NH₂) in the presence of a mild base (e.g., potassium carbonate, piperizine, and morpholine) and an organic solvent (e.g., dimethylformamide (DMF), tetrahydrofuran (THF), ethylacetate (EtOAc), acetonitrile (MeCN), methylene chloride and methanol) produces the 1,4-disubstituted imidazole I(d). Whereas, the 1,4,5-trisubstituted imidazole I(e) may be prepared by reacting the tosylmethyl isocyanide I(c) with the appropriate aldehyde (i.e., R⁴CHO) and the appropriate amine (i.e., R³NH₂) under the same conditions described above (i.e., in the presence of a mild base and an organic solvent). The choice of reaction conditions may vary depending on the solubility of the aldehyde and amine as well as the ease of product isolation. For example, DMF/K₂CO₃ is generally the preferred solvent/base combination; however, other solvent/base combinations may be equally effective and avoid difficulties associated with removing DMF from the product.

Suitable amines for introducing the R³ group into the molecule include methylamine, ethylamine, n-propylamine, iso-propylamine, n-butylamine, sec-butylamine, iso-butylamine, t-butylamine, n-pentylamine, 2-pentylamine, 3-pentylamine, 1,1-dimethyl-propylamine, 3-methylbutylamine, neo-pentylamine, 1,1-dimethyl-3,3-dimethylbutylamine, cyclopropylamine, cyclobutylamine, cyclopentylamine, cyclohexylamine, 1-cyclohexyl-ethylamine, trans-2-benzyloxy-cyclopentylamine, 4-aminomethyl-cyclohexanecarbonitrile, bicyclo[2.2.1]hept-2-ylamine, 1-phenylpropylamine, 2-(4-fluorophenyl)-1,1-dimethylethylamine, 1-p-tolylethylamine, 1-(4-methoxyphenyl)-ethylamine, 1-phenylcyclopentylamine, 1-benzyl-cyclopentylamine, 1-phenyl-cyclohexylamine, 1-benzyl-cyclohexylamine, 2-(4-methoxy-phenoxy)-1,1-dimethyl-ethylamine, 2-(2-methoxy-phenoxy)-1,1-dimethyl-ethylamine, 2-(4-chloro-phenoxy)-1,1-dimethyl-ethylamine, 2-(3-chloro-phenoxy)-1,1-dimethyl-ethylamine, tetrahydropyran-4-ylamine, 2,2-dimethyl-tetrahydropyran-4-ylamine, 1-benzyl-pyrrolidin-3-ylamine, phenylamine, benzylamine, α-phenethylamine, β-phenethylamine, 1,1-dimethylbenzylamine, 2-methylbenzylamine, 2-trifluoroethyl-benzylamine, 2-(4-aminomethyl-phenyl)-propan-2-ol, 2-methoxy-1-phenyl-ethylamine, 1-benzyl-piperidin-4-ylamine, 1-benzyl-piperidin-3-ylamine, indan-1-ylamine, indan-2-ylamine, (1H-indol-4-yl)-methylamine, 5-amino-6-phenyl-piperidin-2-one, 1,1-dioxo-tetrahydrothiophen-3-ylamine, and the like.

Suitable aldehydes for introducing the R⁴ group into the molecule include acetaldehyde, propioaldehyde, n-butrylaldehyde, iso-butrylaldehyde, valeraldehyde, iso-valeraldehyde, pivaldehyde, cyclopentanecarbaldehyde, 2-methylbutanal, caproaldehyde, 2-ethylbutanal, cyclohexylaldehyde, benzaldehyde, 2-phenylpropanal, cuminic aldehyde (4-Isopropyl-benzaldehyde), cinnamaldehyde, salicylaldehyde, m-, o-, or p-methyl-benzaldehyde, mono-, di-, tri-, tetra-substituted halo benzaldehydes, o-, m- or p-anisaldehyde, o-ethoxybenzaldehyde, piperonal, veratraldehyde (3,4-dimethoxybenzaldehyde), p-dimethylaminobenzaldehyde, vanillin (4-hydroxy-3-methoxybenzaldehyde), p-nitrobenzaldehyde, mono-, di- or tri-substituted hydroxybenzaldehydes, furfural, 2-methylfurfural, acrolein, 3-butenal, 2-butenal, glyoxal, hydroxyacetaldehyde, phenoxyacetaldehyde, glyceraldehyde, naphthaldehyde, and the like.

Scheme II illustrates an approach for preparing 1,2,4,5-tetrasubstituted imidazoles (e.g., compounds of Formula (I) where R², R³ and R⁴ are other than hydrogen and X is nitrogen). The synthetic route outlined in Scheme II below is based on the procedures described by N. Coskun and Tirli, F. in Synth. Commun. 27(1), 1(1997) and H. B. Lee and S. Balasubramanian in Org. Lett. 2(3), 323 (2000).

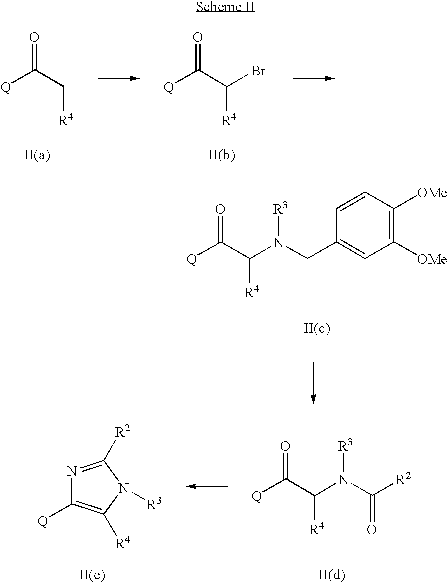

Ketone II(a) where Q is a substituted or unsubstituted 1,5-diphenylpyrazole derivative may be prepared using analogous procedures described in U.S. Pat. Nos. 5,051,518; 5,134,142; and 5,624,941; all of which are incorporated herein by reference, Bischler, Chem. Ber., 26, 1881-1890 (1893), and Tewari, R. S. and P. Parihar, Tetrahedron, 39(1), 129-136 (1983). Other 1,5-disubstituted aryl and heteroaryl pyrazole ketone derivatives may be prepared using analogous procedures. Intermediate II(b) may be prepared using standard bromination procedures well-known to those skilled in the art. For example, bromo compound II(b) may be prepared by treating ketone II(a) with bromine in a chlorinated solvent (e.g., carbon tetrachloride or chloroform) or tetrabutylammonium perbromide in methanol and chloroform. The R³ functionality is introduced into the molecule by reacting the bromo compound II(b) with the appropriate benzyl amine (e.g., N-(3,4-dimethoxybenzyl)-R³amine) in a polar aprotic solvent (e.g., acetonitrile (AcCN)) to produce the benzylic tertiary amine II(c). Preferably, the benzyl group is substituted with electron donating groups to favor the nitrogen-benzylic carbon bond scission in the next step. The benzyl group is cleaved and the R² group introduced into the molecule by treating the benzylic tertiary amine II(c) with the appropriate acid chloride (i.e., R²C(O)Cl) to produce the desired amide II(d). Suitable solvents for the debenzylation/acylation step include anhydrous THF, DMF, 1,2-dichloroethane (DCE) and TMOF. The reaction times and temperatures may vary depending upon the particular solvent used. A preferred solvent is DCE at reflux temperatures. Cyclization to the desired imidazole 11(e) is produced by heating the amide II(d) in the presence of ammonium acetate and glacial acetic acid to about 90° C.

Suitable acid chlorides (i.e., R²C(O)Cl) include formyl chloride, acetyl chloride, n-propionyl chloride, iso-propionyl chloride, n-butyryl chloride, sec-butyryl chloride, iso-butyryl chloride, valeroyl chloride, iso-valeroyl chloride, 2,2-dimethylpropionyl chloride, 2-methylbutyryl chloride, caproyl chloride, 2-ethylbutyryl chloride, 2-methylpentanoyl chloride, 3-methylpentanoyl chloride, 4-methylpentanoyl chloride, 2,2-dimethylbutyryl chloride, 3,3-dimethylbutyryl chloride, 2,3-dimethylbutyryl chloride, n-heptanoyl chloride, 2-methylhexanoyl chloride, 3-methylhexanoyl chloride, 4-methylhexanoyl chloride, 5-methylhexanoyl chloride, 2,2-dimethylpentanoyl chloride, 2,3-dimethylpentanoyl chloride, 3,3-dimethylpentanoyl chloride, 2,4-dimethylpentanoyl chloride, 3,4-dimethylpentanoyl chloride, 4,4-dimethylpentanoyl chloride, 2-ethylpentanoyl chloride, 3-ethylpentanoyl chloride, 2-propylbutyryl chloride, 2-ethyl-3-methylbutyryl chloride, cyclopropylcarbonyl chloride, cyclobutylcarbonyl chloride, cyclopentylcarbonyl chloride, cyclohexylcarbonyl chloride, and the like.

Scheme III illustrates an alternative approach for preparing compounds of Formula (I) where X is nitrogen and R³ is a lone pair of electrons.

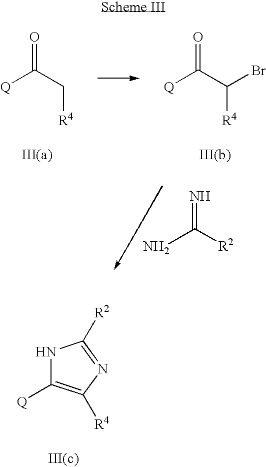

As described above in Scheme II, ketone III(a) where Q is a substituted or unsubstituted 1,5-diphenylpyrazole derivative may be prepared using general procedures described in U.S. Pat. Nos. 5,051,518; 5,134,142; and 5,624,941; all of which are incorporated herein by reference. Other 1,5-disubstituted aryl and heteroaryl pyrazole ketone derivatives may be prepared using analogous procedures. Intermediate III(b) may be prepared using standard bromination procedures well-known to those skilled in the art. For example, bromo compound III(b) may be prepared by treating ketone II(a) with bromine in a chlorinated solvent (e.g., carbon tetrachloride or chloroform) or tetrabutylammonium perbromide in methanol and chloroform. The brominated intermediate III(b) is then reacted with the desired carboxamidine in the presence of a weak base (e.g., potassium carbonate) and chloroform/water to produce the imidazole III(c).

Scheme IV illustrates an approach for preparing compounds of Formula (I) where Y is nitrogen.

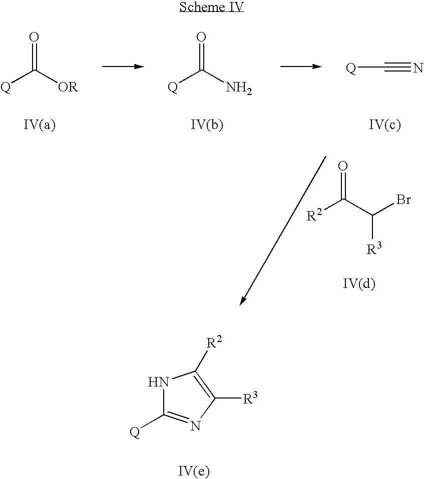

Ester IV(a) where Q is a 1,5-diphenylpyrazole derivative may be prepared using analogous procedures described in U.S. Pat. Nos. 4,944,790, 5,051,518, 5,134,142, and 5,624,941, all of which are incorporated herein by reference, or esterification of the corresponding carboxylic acid prepared by analogous procedures described in Bischler, Chemische Berichte, 26, 1881-1890 (1893). Other 1,5-disubstituted aryl and heteroaryl pyrazole ester derivatives may be prepared using analogous procedures. The corresponding pyrimidine-based esters can be prepared using procedures outlined in: WO9202513. The corresponding imidazole intermediates can be prepared using procedures outlined in: U.S. Pat. No. 5,616,601 (incorporated herein by reference) or C. Gonczi and H. Vander Plas, J. Org. Chem., 46(3), 608-610 (1981). The corresponding triazole intermediates can be prepared using procedures described in: Liebigs Ann. Chem. 48-65 (1984).

Ester IV(a) can be converted to its corresponding amide IV(b) using conventional chemistry well known to those skilled in the art. For example, ester IV(a) is heated in the presence of sodium methoxide and formamide. The amide IV(b) is then converted to the cyano IV (c) by heating the amide IV (b) in the presence of POCl₃. The imidazole IV(e) is formed by reacting cyano derivative IV(c) with ketone IV(d) in the presence of lithium hexamethyidisilamide in an aprotic solvent (e.g., THF) and applying heat.

Conventional methods and/or techniques of separation and purification known to one of ordinary skill in the art can be used to isolate the compounds of the present invention, as well as the various intermediates related thereto. Such techniques will be well-known to one of ordinary skill in is the art and may include, for example, all types of chromatography (high pressure liquid chromatography (HPLC), column chromatography using common adsorbents such as silica gel, and thin-layer chromatography), recrystallization, and differential (i.e., liquid-liquid) extraction techniques.

The compounds of the present invention may be isolated and used per se or in the form of its pharmaceutically acceptable salt, solvate and/or hydrate. The term “salts” refers to inorganic and organic salts of a compound of the present invention. These salts can be prepared in situ during the final isolation and purification of a compound, or by separately reacting the compound, N-oxide, or prodrug with a suitable organic or inorganic acid and isolating the salt thus formed. Representative salts include the hydrobromide, hydrochloride, hydroiodide, sulfate, bisulfate, nitrate, acetate, trifluoroacetate, oxalate, besylate, palmitiate, pamoate, malonate, stearate, laurate, malate, borate, benzoate, lactate, phosphate, hexafluorophosphate, benzene sulfonate, tosylate, formate, citrate, maleate, fumarate, succinate, tartrate, naphthylate, mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, e.g., Berge, et al., J. Pharm. Sci., 66, 1-19 (1977).

The term “prodrug” means a compound that is transformed in vivo to yield a compound of Formula (I) or a pharmaceutically acceptable salt, hydrate or solvate of the compound. The transformation may occur by various mechanisms, such as through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of the present invention contains a carboxylic acid functional group, a prodrug can comprise an ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Similarly, if a compound of the present invention contains an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, P(O)(O(C₁-C₆)alkyl)₂ or glycosyl (the radical resulting from the removal of a hydroxyl group of the hemiacetal form of a carbohydrate).

If a compound of the present invention incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl-natural α-aminoacyl, —C(OH)C(O)OY′ wherein Y′ is H, (C₁-C₆)alkyl or benzyl, —C(OY₀)Y₁ wherein Y₀ is (C₁-C₄) alkyl and Y₁ is (C₁-C₆)alkyl, carboxy(C₁-C₆)alkyl, amino(C₁-C₄)alkyl or mono-N— or di-N,N—(C₁-C₆)alkylaminoalkyl, —C(Y₂)Y₃ wherein Y₂ is H or methyl and Y₃ is mono-N— or di-N,N-(C₁-C₆)alkylamino, morpholino, piperidin-1-yl or pyrrolidin-1-yl.

The compounds of the present invention may contain asymmetric or chiral centers, and, therefore, exist in different stereoisomeric forms. It is intended that all stereoisomeric forms of the compounds of the present invention as well as mixtures thereof, including racemic mixtures, form part of the present invention. In addition, the present invention embraces all geometric and positional isomers. For example, if a compound of the present invention incorporates a double bond or a fused ring, both the cis- and trans- forms, as well as mixtures, are embraced within the scope of the invention. Both the single positional isomers and mixture of positional isomers resulting from the N-oxidation of the pyrimidine and pyrazine rings are also within the scope of the present invention.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Also, some of the compounds of the present invention may be atropisomers (e.g., substituted biaryls) and are considered as part of this invention. Enantiomers can also be separated by use of a chiral HPLC column.

The compounds of the present invention may exist in unsolvated as well as solvated forms with pharmaceutically acceptable solvents such as water, ethanol, and the like, and it is intended that the invention embrace both solvated and unsolvated forms.

It is also possible that the compounds of the present invention may exist in different tautomeric forms, and all such forms are embraced within the scope of the invention. For example, all of the tautomeric forms of the imidazole moiety are included in the invention. Also, for example, all keto-enol and imine-enamine forms of the compounds are included in the invention.

The present invention also embraces isotopically-labeled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine, iodine, and chlorine, such as ²H, ³H, ¹³C, ¹⁴C, ¹⁵N, ¹⁸O, ¹⁷O, ³¹P, ³²P, ³⁵S, ¹⁸F, ¹²I, and ³⁶Cl, respectively.

Certain isotopically-labeled compounds of the present invention (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present invention can generally be prepared by following procedures analogous to those disclosed in the Schemes and/or in the Examples hereinbelow, by substituting an isotopically labeled reagent for a non-isotopically labeled reagent.

Substitution of a halogen group such as chlorine or bromine with iodine is also useful in tracking protein binding of the compound.

Compounds of the present invention are useful for treating diseases, conditions and/or disorders modulated by cannabinoid receptor antagonists; therefore, another embodiment of the present invention is a pharmaceutical composition comprising a therapeutically effective amount of a compound of the present invention and a pharmaceutically acceptable excipient, diluent or carrier.

A typical formulation is prepared by mixing a compound of the present invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The present invention further provides methods of treating diseases, conditions and/or disorders modulated by cannabinoid receptor antagonists in animals that include administering to an animal in need of such treatment a therapeutically effective amount of a compound of the present invention or a pharmaceutical composition comprising an effective amount of a compound of the present invention and a pharmaceutically acceptable excipient, diluent, or carrier. The method is particularly useful for treating diseases, conditions and/or disorders modulated by cannabinoid receptor (in particular, CB1 receptor) antagonists.

Preliminary investigations have indicated that the following diseases, conditions, and/or disorders are modulated by cannabinoid receptor antagonists: eating disorders (e.g., binge eating disorder, anorexia, and bulimia), weight loss or control (e.g., reduction in calorie or food intake, and/or appetite suppression), obesity, depression, atypical depression, bipolar disorders, psychoses, schizophrenia, behavioral addictions, suppression of reward-related behaviors (e.g., conditioned place avoidance, such as suppression of cocaine- and morphine-induced conditioned place preference), substance abuse, addictive disorders, impulsivity, alcoholism (e.g., alcohol abuse, addiction and/or dependence including treatment for abstinence, craving reduction and relapse prevention of alcohol intake), tobacco abuse (e.g., smoking addiction, cessation and/or dependence including treatment for craving reduction and relapse prevention of tobacco smoking), dementia (including memory loss, Alzheimer's disease, dementia of aging, vascular dementia, mild cognitive impairment, age-related cognitive decline, and mild neurocognitive disorder), sexual dysfunction in males (e.g., erectile difficulty), seizure disorders, epilepsy, gastrointestinal disorders (e.g., dysfunction of gastrointestinal motility or intestinal propulsion), attention deficit disorder (ADD including attention deficit hyperactivity disorder (ADHD)), Parkinson's disease, and type II diabetes.

Accordingly, the compounds of the present invention described herein are useful in treating diseases, conditions, or disorders that are modulated by cannabinoid receptor antagonists. Consequently, the compounds of the present invention (including the compositions and processes used therein) may be used in the manufacture of a medicament for the therapeutic applications described herein.

Other diseases, conditions and/or disorders for which cannabinoid receptor antagonists may be effective include: premenstrual syndrome or late luteal phase syndrome, migraines, panic disorder, anxiety, post-traumatic syndrome, social phobia, cognitive impairment in non-demented individuals, non-amnestic mild cognitive impairment, post operative cognitive decline, disorders associated with impulsive behaviours (such as, disruptive behaviour disorders (e.g., anxiety/depression, executive function improvement, tic disorders, conduct disorder and/or oppositional defiant disorder), adult personality disorders (e.g., borderline personality disorder and antisocial personality disorder), diseases associated with impulsive behaviours (e.g., substance abuse, paraphilias and self-mutilation), and impulse control disorders (e.g., intermittene explosive disorder, kleptomania, pyromania, pathological gambling, and trichotillomania)), obsessive compulsive disorder, chronic fatigue syndrome, sexual dysfunction in males (e.g., premature ejaculation), sexual dysfunction in females, disorders of sleep (e.g., sleep apnea), autism, mutism, neurodengenerative movement disorders, spinal cord injury, damage of the central nervous system (e.g., trauma), stroke, neurodegenerative diseases or toxic or infective CNS diseases (e.g., encephalitis or meningitis), cardiovascular disorders (e.g., thrombosis), and diabetes.

The compounds of the present invention can be administered to a patient at dosage levels in the range of from about 0.7 mg to about 7,000 mg per day. For a normal adult human having a body weight of about 70 kg, a dosage in the range of from about 0.01 mg to about 100 mg per kilogram body weight is typically sufficient. However, some variability in the general dosage range may be required depending upon the age and weight of the subject being treated, the intended route of administration, the particular compound being administered and the like. The determination of dosage ranges and optimal dosages for a particular patient is well within the ability of one of ordinary skill in the art having the benefit of the instant disclosure. It is also noted that the compounds of the present invention can be used in sustained release, controlled release, and delayed release formulations, which forms are also well known to one of ordinary skill in the art.

The compounds of this invention may also be used in conjunction with other pharmaceutical agents for the treatment of the diseases, conditions and/or disorders described herein. Therefore, methods of treatment that include administering compounds of the present invention in combination with other pharmaceutical agents are also provided. Suitable pharmaceutical agents that may be used in combination with the compounds of the present invention include anti-obesity agents such as apolipoprotein-B secretion/microsomal triglyceride transfer protein (apo-B/MTP) inhibitors, 11β-hydroxy steroid dehydrogenase-1 (11β-HSD type 1) inhibitors, peptide YY₃₋₃₆ or analogs thereof, MCR-4 agonists, cholecystokinin-A (CCK-A) agonists, monoamine reuptake inhibitors (such as sibutramine), sympathomimetic agents, β₃ adrenergic receptor agonists, dopamine receptor agonists (such as bromocriptine), melanocyte-stimulating hormone receptor analogs, 5HT2c receptor agonists, melanin concentrating hormone antagonists, leptin (the OB protein), leptin analogs, leptin receptor agonists, galanin antagonists, lipase inhibitors (such as tetrahydrolipstatin, i.e. orlistat), anorectic agents (such as a bombesin agonist), Neuropeptide-Y receptor antagonists, thyromimetic agents, dehydroepiandrosterone or an analog thereof, glucocorticoid receptor agonists or antagonists, orexin receptor antagonists, glucagon-like peptide-1 receptor agonists, ciliary neurotrophic factors (such as Axokine™ available from Regeneron Pharmaceuticals, Inc., Tarrytown, N.Y. and Procter & Gamble Company, Cincinnati, Ohio), human agouti-related protein (AGRP) inhibitors, ghrelin receptor antagonists, histamine 3 receptor antagonists or inverse agonists, neuromedin U receptor agonists and the like. Other anti-obesity agents, including the preferred agents set forth hereinbelow, are well known, or will be readily apparent in light of the instant disclosure, to one of ordinary skill in the art.

Especially preferred are anti-obesity agents selected from the group consisting of orlistat, sibutramine, bromocriptine, ephedrine, leptin, peptide YY₃₋₃₆ or an analog thereof, and pseudoephedrine. Preferably, compounds of the present invention and combination therapies are administered in conjunction with exercise and a sensible diet.

Representative anti-obesity agents for use in the combinations, pharmaceutical compositions, and methods of the invention can be prepared using methods known to one of ordinary skill in the art, for example, sibutramine can be prepared as described in U.S. Pat. No. 4,929,629; bromocriptine can be prepared as described in U.S. Pat. Nos. 3,752,814 and 3,752,888; orlistat can be prepared as described in U.S. Pat. Nos. 5,274,143; 5,420,305; 5,540,917; and 5,643,874; and PYY₃₋₃₆ (including analogs) can be prepared as described in U.S. Publication Ser. No. 2002/0,141,985 and WO 03/027637. All of the above recited references are incorporated herein by reference.

Other suitable pharmaceutical agents that may be administered in combination with the compounds of the present invention include agents designed to treat tobacco abuse (e.g., nicotine receptor partial agonists, bupropion hypochloride (also known under the tradename Zyban™) and nicotine replacement therapies), agents to treat erectile dysfunction (e.g., dopaminergic agents, such as apomorphine), ADD/ADHD agents (e.g., Ritalin™, Strattera™, Concerta™ and Adderall™), and agents to treat alcoholism, such as opioid antagonists (e.g., naltrexone (also known under the tradename ReVia™) and nalmefene), disulfiram (also known under the tradename Antabuse™), and acamprosate (also known under the tradename Campral™)). In addition, agents for reducing alcohol withdrawal symptoms may also be co-administered, such as benzodiazepines, beta-blockers, clonidine, carbamazepine, pregabalin, and gabapentin (Neurontin™). Treatment for alcoholism is preferably administered in combination with behavioral therapy including such components as motivational enhancement therapy, cognitive behavioral therapy, and referral to self-help groups, including Alcohol Anonymous (AA).

Other pharmaceutical agents that may be useful include antihypertensive agents; antidepressants (e.g., fluoxetine hydrochloride (Prozac™)); cognitive improvement agents (e.g., donepezil hydrochloride (Aircept™) and other acetylcholinesterase inhibitors); neuroprotective agents (e.g., memantine); antipsychotic medications (e.g., ziprasidone (Geodon™), risperidone (Risperdal™), and olanzapine (Zyprexa™)); insulin and insulin analogs (e.g., LysPro insulin); GLP-1 (7-37) (insulinotropin) and GLP-1 (7-36)-NH₂; sulfonylureas and analogs thereof: chlorpropamide, glibenclamide, tolbutamide, tolazamide, acetohexamide, Glypizide®, glimepiride, repaglinide, meglitinide; biguanides: metformin, phenformin, buformin; α2-antagonists and imidazolines: midaglizole, isaglidole, deriglidole, idazoxan, efaroxan, fluparoxan; other insulin secretagogues: linogliride, A-4166; glitazones: ciglitazone, Actos® (pioglitazone), englitazone, troglitazone, darglitazone, Avandia® (BRL49653); fatty acid oxidation inhibitors: clomoxir, etomoxir; α-glucosidase inhibitors: acarbose, miglitol, emiglitate, voglibose, MDL-25,637, camiglibose, MDL-73,945; β-agonists: BRL 35135, BRL 37344, RO 16-8714, ICI D7114, CL 316,243; phosphodiesterase inhibitors: L-386,398; lipid-lowering agents: benfluorex: fenfluramine; vanadate and vanadium complexes (e.g., Naglivan®) and peroxovanadium complexes; amylin antagonists; glucagon antagonists; gluconeogenesis inhibitors; somatostatin analogs; antilipolytic agents: nicotinic acid, acipimox, WAG 994, pramlintide (Symlin™), AC 2993, nateglinide, aldose reductase inhibitors (e.g., zopolrestat), glycogen phosphorylase inhibitors, sorbitol dehydrogenase inhibitors, sodium-hydrogen exchanger type 1 (NHE-1) inhibitors and/or cholesterol biosynthesis inhibitors or cholesterol absorption inhibitors, especially a HMG-CoA reductase inhibitor, or a HMG-CoA synthase inhibitor, or a HMG-COA reductase or synthase gene expression inhibitor, a CETP inhibitor, a bile acid sequesterant, a fibrate, an ACAT inhibitor, a squalene synthetase inhibitor, an anti-oxidant or niacin. The compounds of the present invention may also be administered in combination with a naturally occurring compound that acts to lower plasma cholesterol levels. Such naturally occurring compounds are commonly called nutraceuticals and include, for example, garlic extract, Hoodia plant extracts, and niacin.

The dosage of the additional pharmaceutical agent will also be generally dependent upon a number of factors including the health of the subject being treated, the extent of treatment desired, the nature and kind of concurrent therapy, if any, and the frequency of treatment and the nature of the effect desired. In general, the dosage range of an anti-obesity agent is in the range of from about 0.001 mg to about 100 mg per kilogram body weight of the individual per day, preferably from about 0.1 mg to about 10 mg per kilogram body weight of the individual per day. However, some variability in the general dosage range may also be required depending upon the age and weight of the subject being treated, the intended route of administration, the particular anti-obesity agent being administered and the like. The determination of dosage ranges and optimal dosages for a particular patient is also well within the ability of one of ordinary skill in the art having the benefit of the instant disclosure.

According to the methods of the invention, a compound of the present invention or a combination of a compound of the present invention and at least one additional pharmaceutical agent is administered to a subject in need of such treatment, preferably in the form of a pharmaceutical composition. In the combination aspect of the invention, the compound of the present invention and at least one other pharmaceutical agent may be administered either separately or in the pharmaceutical composition comprising both. It is generally preferred that such administration be oral. However, if the subject being treated is unable to swallow, or oral administration is otherwise impaired or undesirable, parenteral or transdermal administration may be appropriate.

According to the methods of the invention, when a combination of a compound of the present invention and at least one other pharmaceutical agent are administered together, such administration can be sequential in time or simultaneous with the simultaneous method being generally preferred. For sequential administration, a compound of the present invention and the additional pharmaceutical agent can be administered in any order. It is generally preferred that such administration be oral. It is especially preferred that such administration be oral and simultaneous. When a compound of the present invention and the additional pharmaceutical agent are administered sequentially, the administration of each can be by the same or by different methods.

According to the methods of the invention, a compound of the present invention or a combination of a compound of the present invention and at least one additional pharmaceutical agent (referred to herein as a “combination”) is preferably administered in the form of a pharmaceutical composition. Accordingly, a compound of the present invention or a combination can be administered to a patient separately or together in any conventional oral, rectal, transdermal, parenteral, (for example, intravenous, intramuscular, or subcutaneous) intracisternal, intravaginal, intraperitoneal, intravesical, local (for example, powder, ointment or drop), or buccal, or nasal, dosage form.

Compositions suitable for parenteral injection generally include pharmaceutically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Prevention of microorganism contamination of the compositions can be accomplished with various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents capable of delaying absorption, for example, aluminum monostearate and gelatin.

Solid dosage forms for oral administration include capsules, tablets, powders, and granules. In such solid dosage forms, a compound of the present invention or a combination is admixed with at least one inert customary pharmaceutical excipient (or carrier) such as sodium citrate or dicalcium phosphate or (a) fillers or extenders (e.g., starches, lactose, sucrose, mannitol, silicic acid and the like); (b) binders (e.g., carboxymethylcellulose, alginates, gelatin, polyvinylpyrrolidone, sucrose, acacia and the like); (c) humectants (e.g., glycerol and the like); (d) disintegrating agents (e.g., agar-agar, calcium carbonate, potato or tapioca starch, alginic acid, certain complex silicates, sodium carbonate and the like); (e) solution retarders (e.g., paraffin and the like); (f) absorption accelerators (e.g., quaternary ammonium compounds and the like); (g) wetting agents (e.g., cetyl alcohol, glycerol monostearate and the like); (h) adsorbents (e.g., kaolin, bentonite and the like); and/or (i) lubricants (e.g., talc, calcium stearate, magnesium stearate, solid polyethylene glycols, sodium lauryl sulfate and the like). In the case of capsules and tablets, the dosage forms may also comprise buffering agents.

Solid compositions of a similar type may also be used as fillers in soft or hard filled gelatin capsules using such excipients as lactose or milk sugar, as well as high molecular weight polyethylene glycols, and the like.

Solid dosage forms such as tablets, dragees, capsules, and granules can be prepared with coatings and shells, such as enteric coatings and others well known in the art. They may also contain opacifying agents, and can also be of such composition that they release the compound of the present invention and/or the additional pharmaceutical agent in a delayed manner. Examples of embedding compositions that can be used are polymeric substances and waxes. The drug can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the compound of the present invention or the combination, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed oil, groundnut oil, corn germ oil, olive oil, castor oil, sesame seed oil and the like), glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

Suspensions, in addition to the compound of the present invention or the combination, may further comprise suspending agents, e.g., ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal or vaginal administration preferably comprise suppositories, which can be prepared by mixing a compound of the present invention or a combination with suitable non-irritating excipients or carriers, such as cocoa butter, polyethylene glycol or a suppository wax which are solid at ordinary room temperature but liquid at body temperature and therefore melt in the rectum or vaginal cavity thereby releasing the active component(s).

Dosage forms for topical administration of the compounds of the present invention and combinations may comprise ointments, powders, sprays and inhalants. The drugs are admixed under sterile condition with a pharmaceutically acceptable carrier, and any preservatives, buffers, or propellants that may be required. Ophthalmic formulations, eye ointments, powders, and solutions are also intended to be included within the scope of the present invention.

The following paragraphs describe exemplary formulations, dosages, etc. useful for non-human animals. The administration of the compounds of the present invention and combinations can be effected orally or non-orally (e.g., by injection).

An amount of a compound of the present invention or combination is administered such that an effective dose is received. Generally, a daily dose that is administered orally to an animal is between about 0.01 and about 1,000 mg/kg of body weight, preferably between about 0.01 and about 300 mg/kg of body weight.

Conveniently, a compound of the present invention or combination can be carried in the drinking water so that a therapeutic dosage of the compound is ingested with the daily water supply. The compound can be directly metered into drinking water, preferably in the form of a liquid, water-soluble concentrate (such as an aqueous solution of a water-soluble salt).

Conveniently, a compound of the present invention or combination can also be added directly to the feed, as such, or in the form of an animal feed supplement, also referred to as a premix or concentrate. A premix or concentrate of the compound in a carrier is more commonly employed for the inclusion of the agent in the feed. Suitable carriers are liquid or solid, as desired, such as water, various meals such as alfalfa meal, soybean meal, cottonseed oil meal, linseed oil meal, corncob meal and corn meal, molasses, urea, bone meal, and mineral mixes such as are commonly employed in poultry feeds. A particularly effective carrier is the respective animal feed itself; that is, a small portion of such feed. The carrier facilitates uniform distribution of the compound in the finished feed with which the premix is blended. Preferably, the compound is thoroughly blended into the premix and, subsequently, the feed. In this respect, the compound may be dispersed or dissolved in a suitable oily vehicle such as soybean oil, corn oil, cottonseed oil, and the like, or in a volatile organic solvent and then blended with the carrier. It will be appreciated that the proportions of compound in the concentrate are capable of wide variation since the amount of the compound in the finished feed may be adjusted by blending the appropriate proportion of premix with the feed to obtain a desired level of compound.

High potency concentrates may be blended by the feed manufacturer with proteinaceous carrier such as soybean oil meal and other meals, as described above, to produce concentrated supplements, which are suitable for direct feeding to animals. In such instances, the animals are permitted to consume the usual diet. Alternatively, such concentrated supplements may be added directly to the feed to produce a nutritionally balanced, finished feed containing a therapeutically effective level of a compound of the present invention. The mixtures are thoroughly blended by standard procedures, such as in a twin shell blender, to ensure homogeneity.

If the supplement is used as a top dressing for the feed, it likewise helps to ensure uniformity of distribution of the compound across the top of the dressed feed.

Drinking water and feed effective for increasing lean meat deposition and for improving lean meat to fat ratio are generally prepared by mixing a compound of the present invention with a sufficient amount of animal feed to provide from about 10⁻³ to about 500 ppm of the compound in the feed or water.

The preferred medicated swine, cattle, sheep and goat feed generally contain from about 1 to about 400 grams of a compound of the present invention (or combination) per ton of feed, the optimum amount for these animals usually being about 50 to about 300 grams per ton of feed.

The preferred poultry and domestic pet feeds usually contain about 1 to about 400 grams and preferably about 10 to about 400 grams of a compound of the present invention (or combination) per ton of feed.

For parenteral administration in animals, the compounds of the present invention (or combination) may be prepared in the form of a paste or a pellet and administered as an implant, usually under the skin of the head or ear of the animal in which increase in lean meat deposition and improvement in lean meat to fat ratio is sought.

In general, parenteral administration involves injection of a sufficient amount of a compound of the present invention (or combination) to provide the animal with about 0.01 to about 20 mg/kg/day of body weight of the drug. The preferred dosage for poultry, swine, cattle, sheep, goats and domestic pets is in the range of from about 0.05 to about 10 mg/kg/day of body weight of drug.

Paste formulations can be prepared by dispersing the drug in a pharmaceutically acceptable oil such as peanut oil, sesame oil, corn oil or the like.

Pellets containing an effective amount of a compound of the present invention, pharmaceutical composition, or combination can be prepared by admixing a compound of the present invention or combination with a diluent such as carbowax, carnuba wax, and the like, and a lubricant, such as magnesium or calcium stearate, can be added to improve the pelleting process.

It is, of course, recognized that more than one pellet may be administered to an animal to achieve the desired dose level which will provide the increase in lean meat deposition and improvement in lean meat to fat ratio desired. Moreover, implants may also be made periodically during the animal treatment period in order to maintain the proper drug level in the animal's body.

The present invention has several advantageous veterinary features. For the pet owner or veterinarian who wishes to increase leanness and/or trim unwanted fat from pet animals, the instant invention provides the means by which this may be accomplished. For poultry and swine breeders, utilization of the method of the present invention yields leaner animals that command higher sale prices from the meat industry.

Embodiments of the present invention are illustrated by the following Examples. It is to be understood, however, that the embodiments of the invention are not limited to the specific details of these Examples, as other variations thereof will be known, or apparent in light of the instant disclosure, to one of ordinary skill in the art.

### EXAMPLES

Unless specified otherwise, starting materials are generally available from commercial sources such as Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), and AstraZeneca Pharmaceuticals (London, England).

### General Experimental Procedures

NMR spectra were recorded on a Varian Unity™ 400 (available from Varian Inc., Palo Alto, Calif.) at room temperature at 400 MHz for proton. Chemical shifts are expressed in parts per million (δ) relative to residual solvent as an internal reference. The peak shapes are denoted as follows: s, singlet; d, doublet; t, triplet; q, quartet; m, multiplet; bs, broad singlet; 2s, two singlets. Atmospheric pressure chemical ionization mass spectra (APCI) were obtained on a Fisons™ Platform II Spectrometer (carrier gas: acetonitrile: available from Micromass Ltd, Manchester, UK). Chemical ionization mass spectra (Cl) were obtained on a Hewlett-Packard™ 5989 instrument (ammonia ionization, PBMS: available from Hewlett-Packard Company, Palo Alto, Calif.). Electrospray ionization mass spectra (ES) were obtained on a Waters™ ZMD instrument (carrier gas: acetonitrile: available from Waters Corp., Milford, Mass.). Where the intensity of chlorine or bromine-containing ions are described, the expected intensity ratio was observed (approximately 3:1 for ³⁵Cl/³⁷Cl-containing ions and 1:1 for ⁷⁹Br/⁸¹Br-containing ions) and the intensity of only the lower mass ion is given. In some cases only representative ¹H NMR peaks are given. MS peaks are reported for all examples. Optical rotations were determined on a PerkinElmer™ 241 polarimeter (available from PerkinElmer Inc., Wellesley, Mass.) using the sodium D line (λ=589 nm) at the indicated temperature and are reported as follows [α]_{D}^{temp}, concentration (c=g/100 ml), and solvent.

Column chromatography was performed with either Baker™ silica gel (40 μm; J. T. Baker, Phillipsburg, N.J.) or Silica Gel 50 (EM Sciences™, Gibbstown, N.J.) in glass columns or in Flash 40 Biotage™ columns (ISC, Inc., Shelton, Conn.) under low nitrogen pressure.

The compounds in Example 1 were prepared using the synthetic route generally described in Scheme I above.

### Example 1

### [5-(4-Chloro-phenyl)-1-(2-chloro-phenyl)-4-methyl-1H-pyrazol-3-yl]-methanol (1-a)

To the solution of 5-(4-chloro-phenyl)-1-(2-chloro-phenyl)-4-methyl-1H-pyrazole-3-carboxylic acid ethyl ester (10 g, 26.6 mmol) in toluene (75 ml) was added diisobutylaluminum hydride (44.4 ml of 1.5 M in toluene, 66.6 mmol) at −78° C. The reaction was stirred at −78° C. for 20 minutes and at room temperature for another 2 hours. The reaction mixture was then cooled down to −10° C. Na₂SO₄.10H₂O was added portionwise as a solid over a period of 5 minutes. After additional 10 minute stirring, the cooling was removed and the slurry was stirred for another 45 minutes. The reaction mixture was then diluted with ethyl acetate (100 ml), filtered and washed with ethyl acetate. The filtrate was concentrated in vacuo to give the title compound 1-a as a solid (8.53 g).

### 5-(4-Chloro-phenyl)-1-(2-chloro-phenyl)-4-methyl-1H-pyrazole-3-carbaldehyde (1-b)

To a −78° C. solution of oxalyl chloride (2.9 ml, 33.2 mmol) in methylene chloride (100 ml) was added the DMSO (4.0 ml, 56.1 mmol) over a period of 3 minutes followed by the addition of a solution of [5-(4-chloro-phenyl)-1-(2-chloro-phenyl)-4-methyl-1H-pyrazol-3-yl]-methanol 1-a (8.5 g, 25.5 mmol) in CH₂Cl₂ (50 ml) over a period of 5 minutes. The slurry was stirred for 25 minutes. Triethyl amine (17.8 ml, 128 mmol) was added. The reaction mixture was stirred at −78° C. for another 20 minutes and warmed up to −10° C. Then, the reaction mixture was poured into ether/hexane (1:1, 400 ml), washed with water (200 ml), dried over sodium sulfate and concentrated in vacuo to give the title compound 1-b (8.36 g).

### N-[[5-(4-Chloro-phenyl)-1-(2-chloro-phenyl)-4-methyl-1H-pyrazol-3-yl]-(toluene-4-sulfonyl)-methyl]-formamide (1-c):

To the solution of 5-(4-chloro-phenyl)-1-(2-chloro-phenyl)-4-methyl-1H-pyrazole-3-carbaldehyde 1-b (8.35 g, 25.2 mmol) in acetonitrile (15 ml)/toluene (15 ml) were added formamide (2.5 ml, 63.0 mmol) and chlorotrimethylsilane (3.52 ml, 27.7 mmol). The reaction mixture was stirred at 50° C. for 4 hours, p-toluenesulfinic acid (5.91 g, 37.8 mmol) was added at room temperature and then the reaction mixture was stirred at −50° C. for another 4 hours. Upon the completion of the reaction, the reaction mixture was partitioned with ethyl acetate and water, washed with brine, dried over sodium sulfate and concentrated in vacuo. The residue was purified by a plug of silica gel (600 g, 20%-55% ethyl acetate/hexane) to give the title compound 1-c as a gold foam (12.9 g, 25.2 mmol).

### [[5-(4-Chloro-phenyl)-1-(2-chioro-phenyl-4-methyl-1H-pyrazol-3-yl]-(toluene-4-sulfonyl)-methyl]-methylene-amine (1-d)

Phosphorus oxychloride (2.2 ml, 24 mmol) was added to a solution of N-[[5-(4-chloro-phenyl)-1-(2-chloro-phenyl)-4-methyl-1H-pyrazol-3-yl]-(toluene-4-sulfonyl)-methyl]-formamide 1-c (6.17 g, 12.0 mmol) in THF (48 ml) over a period of 5 minutes. The resultant golden solution was stirred at room temperature for 45 minutes. The reaction was then cooled to −10° C. and 2,6-lutidine (8.4 ml, 72 mmol) was added dropwise over a period of 15 minutes. After another 15 minute stirring, the cooling bath was removed and the reaction mixture was stirred at room temperature for 18 hours. A solution of 40 ml of saturated NaHCO₃ and ice (40 g) was added to the reaction mixture, followed by ethyl acetate (150 ml) and the resultant biphasic mixture was stirred for 15 minutes. The layers were separated and the aqueous layer was extracted with ethyl acetate. The organic layers were combined, washed with 1 N HCl (40 ml), water (40 ml), saturated NaHCO₃ (50 ml), brine, dried over sodium sulfate and concentrated in vacuo to give the title compound 1-d as a dark foam (6.14 g).

### 5-(4-Chloro-phenyl)-1-(2-chloro-ohenyl)-4-methyl-3-[1-(2-trifluoromethyl-benzyl)-1H-imidazol-4-yl]-1H-pvrazole, hydrochloride (1A)

To the slurry of K₂CO₃ (70 mg, 0.5 mmol) in 1 ml dry DMF was added 2-trifluoromethyl-benzylamine hydrochloride (88 mg, 0.5 mmol) followed by glyoxylic acid (46 g, 0.5 mmol). The reaction mixture was stirred for 30 hours at room temperature. [[5-(4-chloro-phenyl)-1-(2-chloro-phenyl)-4-methyl-1H-pyrazol-3-yl]-(toluene-4-sulfonyl)-methyl]-methylene-amine 1-d (125 mg, 0.25 mmol) was then added and the stirring was continued for another 18 hours. The reaction mixture was partitioned with ethyl acetate and water. The organic layer was washed with brine, dried over sodium sulfate and concentrated. The residue was further purified by HPLC (30×50 mm column, 15%-100% AcCN/H₂O) to give the title compound 1A. The product was treated with HCl/ether to form HCl salt as a yellow solid (48 mg).

ms (LCMS) m/z=527.1(M+1) ¹ H NMR in CDCl₃ (ppm): δ 8.85 (1 H, s), 7.87 (2H, m), 7.72 (1 H, t), 7.62 (1 H, t), 7.46 (5H, m), 7.33 (2H, d), 7.20 (2H, d), 5.71 (2H, s), 2.22 (3H, s).

The compounds listed in Table 1-A were prepared using the appropriate starting materials and procedures analogous to those described above for the synthesis of Compound 1A.

TABLE 1-A

| Exam- | | LCMS |
|---|---|---|
| ple | | m/z |
| No. | Compound Name | (M + 1) |
| 1A-1 | 5-(4-Chlorophenyl)-3-(1-cyclopentyl-1H-imidazol- | 471.3 |
| | 4-yl)-1-(2,4-dichlorophenyl)-4-methyl-1H-pyrazole | |
| 1A-2 | 3-(1-Benzyl-1H-imidazol-4-yl)-5-(4-chlorophenyl)- | 493.3 |
| | 1-(2,4-dichlorophenyl)-4-methyl-1H-pyrazole | |
| 1A-3 | 5-(4-Chlorophenyl)-1-(2,4-dichlorophenyl)-3-(1- | 447.3 |
| | isopropyl-1H-imidazol-4-yl)-4-methyl-1H-pyrazole | |
| 1A-4 | 5-(4-Chlorophenyl)-1-(2,4-dichlorophenyl)-4- | 487.8 |
| | methyl-3-[1-(tetrahydro-pyran-4-yl)-1H-imidazol-4- | |
| | yl]-1H-pyrazole | |
| 1A-5 | 5-(4-Chlorophenyl)-3-(1-cyclobutyl-1H-imidazol-4- | 459.3 |
| | yl)-1-(2,4-dichlorophenyl)-4-methyl-1H-pyrazole | |
| 1A-6 | 1-Benzyl-4-{4-[5-(4-chlorophenyl)-1-(2,4- | 576.4 |
| | dichlorophenyl)-4-methyl-1H-pyrazol-3-yl]- | 576.4 |
| | imidazol-1-yl}-piperidine | |
| 1A-7 | 5-(4-Chlorophenyl-3-(1-cyclohexyl-1H-imidazol-4- | 485.4 |
| | yl)-1-(2,4-dichlorophenyl)-4-methyl-1H-pyrazole | |
| 1A-8 | 3-(1-Bicyclo[2.2.1]hept-2-yl-1H-imidazol-4-yl)-5-(4- | 497.2 |
| | chlorophenyl)-1-(2 ,4-dichlorophenyl)-4-methyl-1H- | |
| | pyrazole | |
| 1A-9 | 5-(4-Chlorophenyl)-1-(2,4-dichlorophenyl)-4- | 523.2 |
| | methyl-3-[1-(1-methyl-1-phenyl-ethyl)-1H- | |
| | imidazol-4-yl]-1H-pyrazole | |
| 1A-10 | 5-(4-Chlorophenyl)-1-(2,4-dichlorophenyl)-4- | 447.2 |
| | methyl-3-(1-propyl-1H-imidazol-4-yl)-1H-pyrazole | |
| 1A-11 | 5-(4-Chlorophenyl)-1-(2,4-dichlorophenyl)-3-(1- | 435.1 |
| | ethyl-1H-imidazol-4-yl)-4-methyl-1H-pyrazole | |
| 1A-12 | 3-(1-tert-Butyl-1H-imidazol-4-yl)-5-(4- | 459.1 |
| | chlorophenyl)-1-(2,4-dichlorophenyl)-4-methyl-1H- | |
| | pyrazole | |
| 1A-13 | 5-(4-Chlorophenyl)-1-(2,4-dichlorophenyl)-4- | 507.1 |
| | methyl-3-[1-(1(R)-phenylethyl)-1H-imidazol-4-yl]- | |
| | 1H-pyrazole | |
| 1A-14 | 5-(4-Chlorophenyl)-1-(2,4-dichlorophenyl)-4- | 507.1 |
| | methyl-3-[1-(1(S)-phenylethyl)-1H-imidazol-4-yl]- | |
| | 1H-pyrazole. ms (LCMS) m/z = (M + 1) | |
| 1A-15 | 5-(4-Chlorophenyl)-1-(2,4-dichlorophenyl)-3-(1- | 431.2 |
| | isopropyl-1H-imidazol-4-yl)-1H-pyrazole | |
| 1A-16 | 4-Chloro-5-(4-chlorophenyl)-1-(2,4- | 543.1 |
| | dichlorophenyl)-3-[1-(1-methyl-1-phenyl-ethyl)-1H- | |
| | imidazol-4-yl]-1H-pyrazole | |
| 1A-17 | 4-Chloro-5-(4-chlorophenyl)-1-(2,4-dichloro- | 467.1 |
| | phenyl)-3-(1-isopropyl-1H-imidazol-4-yl)-1H- | |
| | pyrazole | |
| 1A-18 | 5-(4-Chlorophenyl)-1-(2,4-dichlorophenyl)-3-[1-(1- | 509.2 |
| | methyl-1-phenylethyl)-1H-imidazol-4-yl]-1H- | |
| | pyrazole | |
| 1A-19 | 1-(2-Chlorophenyl)-5-(4-chlorophenyl)-3-(1- | 411.2 |
| | isopropyl-1H-imidazol-4-yl)-4-methyl-1H-pyrazole | |
| 1A-20 | 1-(2-Chlorophenyl)-5-(4-chlorophenyl)-3-(1- | 411.2 |
| | isopropyl-1H-imidazol-4-yl)-4-methyl-1H-pyrazole | |
| | hydrochloride salt | |
| 1A-21 | 1-(2-Chlorophenyl)-5-(4-chlorophenyl)-4-methyl-3- | 487.2 |
| | [1-(1-methyl-1-phenyl-ethyl)-1H-imidazol-4-yl]-1H- | |
| | pyrazole | |
| 1A-22 | 1-(2-Chlorophenyl)-5-(4-chlorophenyl)-4-methyl-3- | 487.2 |
| | [1-(1-methyl-1-phenyl-ethyl)-1H-imidazol-4-yl]-1H- | |
| | pyrazole hydrochloride salt | |
| 1A-23 | 1-(2-Chloro-4-methyl-phenyl)-5-(4-chloro-phenyl)- | 425.2 |
| | 3-(1-isopropyl-1H-imidazol-4-yl)-4-methyl-1H- | |
| | pyrazole | |
| 1A-24 | 1-(2-Chloro-4-methyl-phenyl)-5-(4-chloro-phenyl)- | 501.2 |
| | 4-methyl-3-[1-(1-methyl-1-phenyl-ethyl)-1H- | |
| | imidazol-4-yl]-1H-pyrazole | |
| 1A-25 | 5-(4-Chloro-phenyl)-1-(2-chloro-phenyl)-3-(1- | 423.2 |
| | cyclobutyl-1H-imidazol-4-yl)-4-methyl-1H-pyrazole | |
| 1A-26 | 1-(2-Chloro-phenyl)-5-(4-chloro-phenyl)-3-[1-(1,1- | 439.2 |
| | dimethyl-propyl)-1H-imidazol-4-yl]-4-methyl-1H- | |
| | pyrazole | |
| 1A-27 | 5-(4-Chloro-phenyl)-1-(2-chloro-phenyl)-3-(1- | 423.2 |
| | cyclopropyl-1H-imidazol-4-yl)-4-methyl-1H- | |
| | pyrazole | |
| 1A-28 | 5-(4-Chloro-phenyl)-1-(2-chloro-phenyl)-4-methyl- | 527.2 |
| | 3-[1-(1-phenyl-cyclohexyl)-1H-imidazol-4-yl]-1H- | |
| | pyrazole | |
| 1A-29 | 3-[1-(1-Benzyl-cyclohexyl)-1H-imidazol-4-yl]-1-(2- | 541.2 |
| | chloro-phenyl)-5-(4-chloro-phenyl)-4-methyl-1H- | |
| | pyrazole | |
| 1A-30 | 3-[1-(1-Benzyl-cyclopentyl)-1H-imidazol-4-yl]-1-(2- | 527.2 |
| | chloro-phenyl)-5-(4-chloro-phenyl)-4-methyl-1H- | |
| | pyrazole | |
| 1A-31 | 1-(2-Chloro-phenyl)-5-(4-chloro-phenyl)-4-methyl- | 481.2 |
| | 3-[1-(1,1,3,3-tetramethyl-butyl)-1H-imidazol-4-yl]- | |
| | 1H-pyrazole | |
| 1A-32 | 5-(4-Chloro-phenyl)-1-(2-chloro-phenyl)-3-(1- | 485.2 |
| | indan-2-yl-1H-imidazol-4-yl)-4-methyl-1H-pyrazole | |
| 1A-33 | 5-(4-Chloro-phenyl)-1-(2-chloro-phenyl)-4-methyl- | 513.2 |
| | 3-[1-(1-phenyl-)-1H-imidazol-4-yl]-1H-pyrazole | |
| 1A-34 | 5-(4-Chloro-phenyl)-1-(2-chloro-phenyl)-4-methyl- | 473.1 |
| | 3-[1-(1-phenyl-ethyl)-1H-imidazol-4-yl]-1H- | |
| | pyrazole | |
| 1A-35 | 5-(4-Chloro-phenyl)-1-(2-chloro-phenyl)-3-(1- | 485.2 |
| | indan-1-yl-1H-imidazol-4-yl)-4-methyl-1H-pyrazole | |
| 1A-36 | 5-(4-Chloro-phenyl)-1-(2-chloro-phenyl)-3-[1-(1- | 479.2 |
| | cyclohexyl-ethyl)-1H-imidazol-4-yl]-4-methyl-1H- | |
| | pyrazole | |
| 1A-37 | 5-(4-Chloro-phenyl)-1-(2-chloro-phenyl)-4-methyl- | 487.2 |
| | 3-[1-(1-phenyl-propyl)-1H-imidazol-4-yl]-1H- | |
| | pyrazole | |
| 1A-38 | 1-(2-Chloro-phenyl)-5-(4-chloro-phenyl)-3-{1-[2-(4- | 519.1 |
| | fluoro-phenyl)-1,1-dimethyl-ethyl]-1H-imidazol-4- | |
| | yl}-4-methyl-1H-pyrazole | |
| 1A-39 | 5-(4-Chloro-phenyl)-1-(2-chloro-phenyl)-3-(1- | 485.2 |
| | indan-1-yl-1H-imidazol-4-yl)-4-methyl-1H-pyrazole | |
| 1A-40 | 5-(4-Chloro-phenyl)-1-(2-chloro-phenyl)-4-methyl- | 487.2 |
| | 3-[1-(1-p-tolyl-ethyl)-1H-imidazol-4-yl]-1H-pyrazole | |
| 1A-41 | 5-(4-Chloro-phenyl)-1-(2-chloro-phenyl)-3-{1-[1-(4- | 503.1 |
| | methoxy-phenyl)-ethyl]-1H-imidazol-4-yl}-4- | |
| | methyl-1H-pyrazole | |
| 1A-42 | 5-(4-Chloro-phenyl)-1-(2-fluoro-phenyl)-3-(1- | 395.2 |
| | isopropyl-1H-imidazol-4-yl)-4-methyl-1H-pyrazole | |
| 1A-43 | 5-(4-Chloro-phenyl)-1-(2-fluoro-phenyl)-4-methyl- | 471.2 |
| | 3-[1-(1-methyl-1-phenyl-ethyl)-1H-imidazol-4-yl]- | |
| | 1H-pyrazole | |
| 1A-44 | 5-(4-Chloro-phenyl)-3-[1-(1,1-dimethyl-propyl)-1H- | Data? |
| | imidazol-4-yl]-1-(2-fluoro-phenyl)-4-methyl-1H- | |
| | pyrazole | |
| 1A-45 | 5-(4-Chloro-phenyl)-1-(2-fluoro-phenyl)-4-methyl- | 457.2 |
| | 3-[1-(1-phenyl-ethyl)-1H-imidazol-4-yl]-1H- | |
| | pyrazole | |
| 1A-46 | 5-(4-Chloro-phenyl)-1-(2-chloro-phenyl)-3-[1-(2,2- | 481.2 |
| | dimethyl-tetrahydro-pyran-4-yl)-1H-imidazol-4-yl]- | |
| | 4-methyl-1H-pyrazole | |
| 1A-47 | 5-(4-Chloro-phenyl)-1-(2-chloro-phenyl)-4-methyl- | 445.2 |
| | 3-(1-phenyl-1H-imidazol-4-yl)-1H-pyrazole. ms | |
| | (LCMS) m/z = (M + 1) | |
| 1A-48 | 5-(4-Chloro-phenyl)-1-(2-chloro-phenyl)-4-methyl- | 527.1 |
| | 3-[1-(2-trifluoromethyl-benzyl)-1H-imidazol-4-yl]- | |
| | 1H-pyrazole | |

### 5-(4-Chloro-phenyl)-1-(2-chloro-phenyl)-4-methyl-3-(1-methyl-5-Phenyl-1H-imidazol-4-yl)-1H-pyrazole, hydrochloride (1B)

To the solution of benzaldehyde (103 μl, 1.0 mmol) in dry THF (1.0 ml) was added methyl amine (400 μl, 0.805 mmol) at room temperature. The reaction mixture was stirred for 1.5 hours and morpholine (105 μl, 1.21 mmol) was added followed by [[5-(4-chloro-phenyl)-1-(2-chloro-phenyl)-4-methyl-1H-pyrazol-3-yl]-(toluene-4-sulfonyl)-methyl]-methylene-amine 1-d (200 mg, 0.402 mmol). The reaction mixture was then left stirring overnight. The solvent was removed in vacuo. The residue was purified by chromatography (silica, 0-5% MeOH/CH₂Cl₂). The product was then treated with HCl/ether to form the title salt 1B as a tan solid (129 mg).

ms (LCMS) m/z=459.1 (M+1) ¹H NMR in CDCl₃ (ppm): δ 9.14 (1H, s), 7.56 (5H, m), 7.47 (2H, q), 7.41 (2H, q), 7.29 (2H, d), 7.10 (2H, d), 3.88 (3H, s), 1.56 (3H, s).

The compounds listed in Table 1-B were prepared using the appropriate starting materials and procedures analogous to those described above for the synthesis of Compound 1 B.

TABLE 1-B

| | | LCMS |
|---|---|---|
| Example | | m/z |
| No. | Compound Name | (M + 1) |
| 1B-1 | 5-(4-Chlorophenyl)-1-(2,4-dichlorophenyl)-3-(1- | 461.2 |
| | isopropyl-5-methyl-1H-imidazol-4-yl)-4-methyl- | |
| | 1H-pyrazole | |
| 1B-2 | 5-(4-Chlorophenyl)-1-(2,4-dichlorophenyl)-3-(5- | 473.2 |
| | ethyl-1-isopropyl-1H-imidazol-4-yl)-4-methyl-1H- | |
| | pyrazole | |
| 1B-3 | {5-[5-(4-Chlorophenyl)-1-(2,4-dichlorophenyl)-4- | 475.2 |
| | methyl-1H-pyrazol-3-yl]-3-isopropyl-3H-imidazol- | |
| | 4-yl}-methanol | |
| 1B-4 | 5-(4-Chloro-phenyl)-1-(2-chloro-phenyl)-3-(1- | 425.2 |
| | isopropyl-5-methyl-1H-imidazol-4-yl)-4-methyl-1H- | |
| | pyrazole | |
| 1B-5 | 5-(4-Chloro-phenyl)-1-(2-chloro-phenyl)-3-(1- | 437.2 |
| | cyclobutyl-5-methyl-1H-imidazol-4-yl)-4-methyl- | |
| | 1H-pyrazole | |
| 1B-6 | 1-(2-Chloro-phenyl)-5-(4-chloro-phenyl)-3-[1-(1- | 453.2 |
| | ethyl-propyl)-5-methyl-1H-imidazol-4-yl]-4- | |
| | methyl-1H-pyrazole | |
| 1B-7 | 1-(2-Chloro-phenyl)-5-(4-chloro-phenyl)-3-(1- | 423.2 |
| | cyclopropyl-5-methyl-1H-imidazol-4-yl)-4-methyl- | |
| | 1H-pyrazole | |
| 1B-8 | 1-(2-Chloro-phenyl)-5-(4-chloro-phenyl)-3-(1- | 499.1 |
| | indan-2-yl-5-methyl-1H-imidazol-4-yl)-4-methyl- | |
| | 1H-pyrazole | |
| 1B-9 | [5-[5-(4-Chloro-phenyl)-1-(2-chloro-phenyl)-4- | 503.2 |
| | methyl-1H-pyrazol-3-yl]-3-(1-phenyl-ethyl)-3H- | |
| | imidazol-4-yl]-methanol. | |
| 1B-10 | 5-(4-Chloro-phenyl)-1-(2-chloro-phenyl)-3-[1- | 515.2 |
| | isopropyl-5-(1-phenyl-ethyl)-1H-imidazol-4-yl]-4- | |
| | methyl-1H-pyrazole | |
| 1B-11 | 1-(2-Chloro-phenyl)-5-(4-chloro-phenyl)-3-(1- | 487.2 |
| | isopropyl-5-phenyl-1H-imidazol-4-yl)-4-methyl- | |
| | 1H-pyrazole. | |
| 1B-12 | 5-(4-Chloro-phenyl)-1-(2-chloro-phenyl)-4-methyl- | 473.2 |
| | 3-[5-(1-phenyl-ethyl)-1H-imidazol-4-yl]-1H- | |
| | pyrazole | |
| 1B-13 | 5-(4-Chloro-phenyl)-1-(2-chloro-phenyl)-4-methyl- | 459.1 |
| | 3-(1-methyl-5-phenyl-1H-imidazol-4-yl)-1H- | |
| | pyrazole | |
| 1B-14 | 1-(2-Chloro-phenyl)-5-(4-chloro-phenyl)-3-[1-(1,1- | 453.2 |
| | dimethyl-propyl)-5-methyl-1H-imidazol-4-yl]-4- | |
| | methyl-1H-pyrazole | |
| 1B-15 | 5-(4-Chloro-phenyl)-1-(2-chloro-phenyl)-4-methyl- | 453.2 |
| | 3-[1-methyl-5-(1-phenyl-ethyl)-1H-imidazol-4-yl]- | |
| | 1H-pyrazole | |
| 1B-16 | 1-(2-Chloro-phenyl)-5-(4-chloro-phenyl)-4-methyl- | 445.1 |
| | 3-(5-phenyl-1H-imidazol-4-yl)-1H-pyrazole | |
| 1B-17 | 5-(4-Chloro-phenyl)-1-(2-chloro-phenyl)-3-(5- | 411.2 |
| | isopropyl-1H-imidazol-4-yl)-4-methyl-1H-pyrazole | |
| 1B-18 | 1-(2-Chloro-phenyl)-5-(4-chloro-phenyl)-4-methyl- | 501.2 |
| | 3-[5-methyl-1-(1-methyl-1-phenyl-ethyl)-1H- | |
| | imidazol-4-yl]-1H-pyrazole | |
| 1B-19 | 5-(4-Chloro-phenyl)-1-(2-chloro-phenyl)-4-methyl- | 487.2 |
| | 3-[5-methyl-1-(1-phenyl-ethyl)-1H-imidazol-4-yl]- | |
| | 1H-pyrazole | |

The compounds in Example 2 were prepared using the synthetic route generally described in Scheme IlIl above.

### Example 2

### 2-Bromo-1-[5-(4-chlorophenyl)-1-(2,4-dichlorophenyl)-4-methyl-1H-pyrazol-3-yl]-ethanone (I-2a)

1-[5-(4-Chlorophenyl)-1-(2,4-dichlorophenyl)-4-methyl-1H-pyrazol-3-yl]-ethanone (569 mg, 1.5 mM) was dissolved in CHCl₃ (5 ml) and to it was added bromine (81 μl, 1.575 mM) dropwise. The reaction mixture was stirred overnight at room temperature and then washed with saturated NaHCO₃ solution and then brine. The organic layer was dried (Na₂SO₄), filtered, and concentrated to give the title compound (I-2a) as a white foam (599 mg).

### 5-(4-Chlorophenyl)-3-(2-cyclohexyl-3H-imidazol-4-yl)-1-(2,4-dichlorophenyl)-4-methyl-1H-pyrazole (2A)

2-Bromo-1-[5-(4-chlorophenyl)-1-(2,4-dichlorophenyl)-4-methyl-1H-pyrazol-3-yl]-ethanone I-2a (200 mg, 0.44 mM) and cyclohexane carboxamidine (71 mg, 0.44 mM) were combined in CH₂Cl₂ (2 ml). To this mixture was added aqueous K₂CO₃ (1 ml, 30% w/w) and it was stirred at room temperature overnight. The reaction had not gone to completion per TLC so it was heated to 50° C. overnight. The completed reaction was cooled to room temperature and partitioned between ethyl acetate and water. The organic layer was washed with water then brine. The organic layer was dried (Na₂SO₄), filtered, and concentrated to dryness. The crude product was purified via silica gel chromatography (gradient of 30% to 40% ethyl acetate/hexanes) to give the title compound 2A as a white solid (26 mg);

ms (LCMS) m/z=487.2 (M+1). ¹H NMR in CD₂Cl₂ (ppm): δ 7.42-7.1 (m, 8H)), 2.76 (m,1 H)), 2.24 (s, 3H), 2.1-1.2 (m, 10H).

### 5-(4-Chlorophenyl)-1-(2,4-dichlorophenyl)-3-(2-isopropyl-3H-imidazol-4-yl)-4-methyl-1H-pyrazole hydrochloride salt (2B)

5-(4-chlorophenyl)-1-(2,4-dichlorophenyl)-3-(2-isopropyl-3H-imidazol-4-yl)-4-methyl-1H-pyrazole was prepared using analogous procedures as described above for the synthesis of compound 2A. The HCl salt was prepared by dissolving 5-(4-chlorophenyl)-1-(2,4-dichlorophenyl)-3-(2-isopropyl-3H-imidazol-4-yl)-4-methyl-1H-pyrazole (47 mg, 0.11 mM) in CH₂Cl₂ (0.5 ml) and cooling the solution to 0° C. To this soliution was added 1M HCl in diethyl ether (0.2 ml, 2 equiv.) and the mixture warmed to room temperature. The reaction was concentrated to dryness and pumped on high vacuum to afford the title compound 3B as an off-white solid (36 mg);

ms (LCMS) m/z=445.2(M+1). ¹H NMR in CD₂Cl₂ (ppm): δ 7.52 (s, 1H), 7.45 (s, 1H), 7.34-7.32 (m, 4H), 7.11 (d, 2H), 3.6 (m, 1H), 2.23 (s, 3H), 1.52 (d, 6H).

The compounds in Example 3 were prepared using the synthetic route generally described in Scheme IV above.

### Example 3

### 5-(4-Chiorophenyl)-1-(2,4-dichloroohenyl)-4-methyl-1H-pyrazole-3-carboxylic acid amide (I-3a):

5-(4-Chloro-phenyl)-1-(2,4-dichloro-phenyl)-4-methyl-1H-pyrazole-3-carboxylic acid methyl ester (2.5 g, 6.32 mM) and sodium methoxide (1.04 g, 19.28 mM) were combined in formamide (12 ml) and heated to 100° C. overnight. The reaction mixture was cooled to room temperature and the crude product was filtered off and washed with water. The crude material was purified by silica gel chromatography (gradient of 40% to 60% ethyl acetate/hexanes) to yield the title compound I-3a as a white solid (1.05 g); ms (LCMS) m/z=380.1 (M+1).

### 5-(4-Chloro-phenyl)-1-(2,4-dichloro-phenyl)-4-methyl-1H-pyrazole-3-carbonitrile (I-3b)

5-(4-Chloro-phenyl)-1-(2,4-dichloro-phenyl)-4-methyl-1H-pyrazole-3-carboxylic acid amide I-3a (1.05 g, 2.76 mM) was dissolved in phosphorous oxychloride (5 ml) and refluxed for an hour. The reaction mixture was poured into cool water and stirred for 30 minutes. The aqueous solution was extracted with diethyl ether. The organic layer was washed with brine, dried (Na₂SO₄), filtered, and concentrated to dryness to afford the title compound I-3b (956 mg); ms (LCMS) m/z=364.1 (M+1).

### 5-(4-Chloro-phenyl)-3-(5-cyclohexyl-1H-imidazol-2-yl)-1-(2,4-dichloro-phenyl)-4-methyl-1H-pyrazole (3A)

5-(4-Chloro-phenyl)-1-(2,4-dichloro-phenyl)-4-methyl-1H-pyrazole-3-carbonitrile I-3b (250 mg, 0.69 mM) was dissolved in tetrahydrofuran (3 ml) and cooled to 0° C. Lithium bis(trimethylsilyl)amide (0.83 ml of 1.0M in THF) was added dropwise and the mixture warmed to room temperature. A TLC after 4 hours showed reaction not complete therefore the reaction was warmed with a hot water bath for 2 hours. The reaction mixture was cooled to room temperature and to it was added NaHCO₃ (175 mg in 3 ml of water). Then 2-bromo-1-cyclohexyl-ethanone (141 mg, 0.69 mM in 3 ml CHCl₃) was added to the reaction mixture and it was stirred at room temperature for 72 hours. The reaction mixture was partitioned between ethyl acetate/water. The organic layers were combined, washed with brine, dried (Na₂SO₄), filtered, and concentrated to dryness. The crude product was purified via silica gel chromatography (gradient of 15% to 20% ethyl acetate/ hexanes) to obtain the title compound 3A as a white foam (14 mg);

ms (LCMS) m/z=485.2 (M+1). ¹H NMR in CD₂CI₂ (ppm): δ 7.44 (s, 1 H), 7.32-7.30 (m, 5H), 7.14 (d, 2H), 2.64 (m, 1H), 2.44 (s, 3H), 2.03-1.24 (m, 10H).

The compound listed in Table 2 was prepared using the appropriate starting materials and procedures analogous to those described above for the synthesis of Compound 3A.

TABLE 2

| | | LCMS |
|---|---|---|
| Example | | m/z |
| No. | Compound Name | (M + 1) |
| 3B | 5-(4-Chloro-phenyl)-1-(2,4-dichloro-phenyl)-3-(5- | 445.2 |
| | isopropyl-1H-imidazol-2-yl)-4-methyl-1H-pyrazole | |

### Pharmacological Testing

The utility of the compounds of the present invention in the practice of the instant invention can be evidenced by activity in at least one of the protocols described hereinbelow. The following acronyms are used in the protocols described below.

BSA—bovine serum albumin

DMSO—dimethylsulfoxide

EDTA—ethylenediamine tetracetic acid

PBS—phosphate-buffered saline

EGTA—ethylene glycol-bis(β-aminoethyl ether) N,N,N′,N′-tetraacetic acid

GDP—guanosine diphosphate

sc—subcutaneous

po—orally

ip—intraperitoneal

icv—intra cerebro ventricular

iv—intravenous

[3H]SR141716A-radiolabeled N-(piperidin-1-yl)-5-(4-chlorophenyl)-1-(2,4-dichlorophenyl)-4-methyl-1H-pyrazole-3-carboxamide hydrochloride available from Amersham Biosciences, Piscataway, N.J.

[3H] 5-(1,1-dimethyl-heptyl)-2-[5-hydroxy-2-(3-hydroxy-propyl)-cyclohexyl]-phenol available from NEN Life Science Products, Boston, Mass.

AM251-N-(piperidin-1-yl)-1-(2,4-dichlorophenyl)-5-(4-iodophenyl)-4-methyl-1H-pyrazole-3-carboxamide available from Tocris™, Ellisville, Mo.

All of the compounds listed in the Example section above were tested in the CB-1 receptor binding assay below. Those compounds having an activity <20 nM were then tested in the CB-1 GTPγ [³⁵S] Binding Assay and the CB-2 binding assay described below in the Biological Binding Assays section. Selected compounds were then tested in vivo using one or more of the functional assays described in the Biological Functional Assays section below.

### Biological Binding Assays

Bioassay systems for determining the CB1 and CB2 binding properties and pharmacological activity of cannabinoid receptor ligands are described by Roger G. Pertwee in “Pharmacology of Cannabinoid Receptor Ligands” Current Medicinal Chemistry, 6, 635-664 (1999) and in WO 92/02640 (U.S. application Ser. No. 07/564,075 filed Aug. 8, 1990, incorporated herein by reference).

The following assays were designed to detect compounds that inhibit the binding of [3H] SR141716A (selective CB-1 radiolabeled ligand) and [3H] 5-(1,1-dimethyl-heptyl)-2-[5-hydroxy-2-(3-hydroxy-propyl)-cyclohexyl]-phenol (CB-1/CB-2 radiolabeled ligand) to their respective receptors.

### CB-1 Receptor Binding Protocol

PelFreeze brains (available from Pel Freeze Biologicals, Rogers, Ark.) were cut up and placed in tissue preparation buffer (5 mM Tris HCl, pH=7.4 and 2 mM EDTA), polytroned at high speed and kept on ice for 15 minutes. The homogenate was then spun at 1,000×g for 5 minutes at 4° C. The supernatant was recovered and centrifuged at 100,000×G for 1 hour at 4° C. The pellet was then re-suspended in 25 ml of TME (25 nM Tris, pH=7.4, 5 mM MgCl₂, and 1 mM EDTA) per brain used. A protein assay was performed and 200 μl of tissue totaling 20 μg was added to the assay.

The test compounds were diluted in drug buffer (0.5% BSA, 10% DMSO and TME) and then 25 μl were added to a deep well polypropylene plate. [3H] SR141716A was diluted in a ligand buffer (0.5% BSA plus TME) and 25 μl were added to the plate. A BCA protein assay was used to determine the appropriate tissue concentration and then 200 μl of rat brain tissue at the appropriate concentration was added to the plate. The plates were covered and placed in an incubator at 20° C. for 60 minutes. At the end of the incubation period 250 μl of stop buffer (5% BSA plus TME) was added is to the reaction plate. The plates were then harvested by Skatron onto GF/B filtermats presoaked in BSA (5 mg/ml) plus TME. Each filter was washed twice. The filters were dried overnight. In the morning the filters were counted on a Wallac Betaplate™ counter (available from PerkinElmer Life Sciences™, Boston, Mass.). An activity range from 0.5 to 500 nanomolar was observed for the compounds listed in the Example section above. As a specific example, a binding affinity of 371 nanomolar was observed for the compound of Example 1B-13. Example 1B-13 was chosen for illustrative purposes only and does not imply that the compound of Example 1B-13 is a preferred compound.

### CB-2 Receptor Binding Protocol

CHO cells transfected with CB-2 (obtained from Dr. Debra Kendall, University of Connecticut) were harvested in tissue preparation buffer (5 mM Tris-HCl buffer (pH=7.4) containing 2 mM EDTA), polytroned at high speed and kept on ice for 15 minutes. The homogenate was then spun at 1,000×g for 5 minutes at 4° C. The supernatant was recovered and centrifuged at 100,000×G for 1 hour at 4° C. The pellet was then re-suspended in 25 ml of TME (25 mM Tris buffer (pH=7.4) containing 5 mM MgCl₂ and 1 mM EDTA) per brain used. A protein assay was performed and 200 μl of tissue totaling 10 μg was added to the assay.

The test compounds were diluted in drug buffer (0.5% BSA, 10% DMSO, and 80.5% TME) and then 25 μl were added to the deep well polypropylene plate. [3H] 5-(1,1-dimethyl-heptyl)-2-[5-hydroxy-2-(3-hydroxy-propyl)-cyclohexyl]-phenol was diluted a ligand buffer (0.5% BSA and 99.5% TME) and then 25 μl were added to each well at a concentration of 1 nM. A BCA protein assay was used to determine the appropriate tissue concentration and 200 μl of the tissue at the appropriate concentration was added to the plate. The plates were covered and placed in an incubator at 30° C. for 60 minutes. At the end of the incubation period 250 μl of stop buffer (5% BSA plus TME) was added to the reaction plate. The plates were then harvested by Skatron format onto GF/B filtermats presoaked in BSA (5 mg/ml) plus TME. Each fifter was washed twice. The filters were dried overnight. The filters were then counted on the Wallac Betaplate™ counter.

### CB-1 GTPγ [³⁵S] Binding Assay

Membranes were prepared from HEK293 cells (CRL-1573 available from the American Type Culture Collection (ATCC), Manassas, Va.) stably transfected with the human CB-1 receptor cDNA. Membranes were prepared from cells as described by Bass et al, in “Identification and characterization of novel somatostatin antagonists,” Molecular Pharmacology, 50, 709-715 (1996). GTPγ [³⁵S] binding assays were performed in a 96 well FlashPlate™ format in duplicate using 100 pM GTPγ[³⁵S] and 10 μg membrane per well in assay buffer composed of 50 mM Tris HCl, pH 7.4, 3 mM MgCl₂, pH 7.4, 10 mM MgCl₂, 20 mM EGTA, 100 mM NaCl, 30 μM GDP, 0.1% bovine serum albumin and the following protease inhibitors: 100 μg/ml bacitracin, 100 μg/ml benzamidine, 5 μg/ml aprotinin, 5 μg/ml leupeptin. The assay mix was then incubated with increasing concentrations of antagonist (10⁻¹⁰ M to 10⁻⁵ M) for 10 minutes and challenged with the CB agonist 5-(1,1-dimethyl-heptyl)-2-[5-hydroxy-2-(3-hydroxy-propyl)-cyclohexyl]-phenol (10 μM). Assays were performed at 30° C. for one hour. The FlashPlates™ were then centrifuged at 2000×g for 10 minutes. Stimulation of GTPγ[³⁵S] binding was then quantified using a Wallac Microbeta. EC₅₀ calculations were done using Prism™ by Graphpad.

### Biological Functional Assays

The following in-vivo assay is based on the observation that Δ⁹-tetrahydrocannabinol (Δ⁹-THC) has been shown to decrease general locomotor activity in male ICR mice. Therefore, a reversal in decreased activity by pre-treating with a CB-1 antagonist provides a screen for in-vivo activity.

### Locomotor Activity

Male ICR mice (17-19 g, Charles River Laboratories, Inc., Wilmington, Mass.) were pre-treated with test compound (sc, po, ip, or icv). Ten minutes later, the mice were challenged with Δ⁹-THC. Five minutes after the THC injection, the mice were placed in clear acrylic cages (431.8 cm×20.9 cm×20.3 cm) containing clean wood shavings. The subjects were allowed to explore surroundings for a total of about 5 minutes and the activity was recorded by infrared motion detectors (available from Coulbourn Instruments™, Allentown, Pa.) that were placed on top of the cages. The data was computer collected and expressed as “movement units.”

The data was presented as a percent reversal of the agonist induced decrease in locomotor activity calculated using the following formula.

cp/agonist—vehicle/agonist)/(vehicle/vehicle—vehicle/agonist Negative numbers indicate a potentiation of the agonist activity or non-antagonist activity. Positive numbers indicate a reversal of the hypo-locomotion or antagonist activity.

Cannabinoids have also been shown to produce catalepsy in rodents. Therefore, reversal of catalepsy by pre-treating with a CB-1 antagonist also provides a useful screen for in-vivo activity.

### Catalepsy

Male ICR mice (17-19 g) were pre-treated with test compound (sc, po, ip or icv). Ten minutes later, the mice were challenged with Δ⁹-THC (iv). Ninety minutes post iv injection, the mice were placed on a 6.5 cm steel ring having attached thereto a ring stand at a height of about 12 inches. The ring was mounted in a horizontal orientation and the mouse was suspended in the gap of the ring with fore- and hind-paws gripping the perimeter. The duration that the mouse remains completely motionless (except for respiratory movements) was recorded over a 3-minute period.

The data was presented as a percent immobility rating. The rating was calculated by dividing the number of seconds the mouse remains motionless by the total time of the observation period and multiplying the result by 100. A percent reversal from the agonist was also calculated: (cp/agonist—vehicle/agonist)/(vehicle/vehicle—vehicle/agonist).

### Food Intake

The following screen was used to evaluate the efficacy of test compounds for inhibiting food intake in Sprague-Dawley rats after an overnight fast.

Male Sprague-Dawley rats were obtained from Charles River Laboratories, Inc. (Wilmington, Mass.). The rats were individually housed and fed powdered chow. They were maintained on a 12 hour light/dark cycle and received food and water ad libitum. The animals were given one week to acclimate to the vivarium before testing. Testing was completed during the light portion of the cycle.

Food was removed from the cages the afternoon of the day prior to testing and the rats were fasted overnight. After the overnight fast, the rats were dosed with vehicle or test compounds. A known antagonist was dosed (3 mg/kg) as a positive control. The test compounds were dosed at ranges between 0.1 and 100 mg/kg depending upon the compound. The standard vehicle was 30% β-cyclodextrin in water and the stand route of administration was p.o. However, different vehicles and routes of administration may be used to accommodate various compounds. The rats were weighed and the body weights recorded at the time of dosing. Food was re-introduced 30 minutes after dosing. Food weights were then taken at 2 hours, 4 hours and 24 hours post-reintroduction of food. Paper was placed under the food jars to collect spillage, and weighed at each time-point. Body weights were recorded again at 24 hours post-food re-introduction.

The following assay was used to identify reverse hypothermia in mice.

### Hypothermia

Male ICR mice (17-19 g) were pretreated (N=7/treatment) with test compounds (sc, po, ip or icv). Ten minutes later, mice were challenged with a is CB-1 agonist (sc, po, iv or ip). At various time periods after the agonist, rectal body temperatures were taken.

Data was presented as a percent reversal of the agonist-induced hypothermia. This number was calculated by taking the mean body temperature of the test compound/agonist group minus the mean of the vehicle/agonist group over the mean of the vehicle/vehicle group minus the mean of the vehicle/agonist group. Negative numbers indicate a potentiation of the agonist-induced hypothermia; whereas, positive numbers indicate a reversal of the hypothermic effect.

### Detection of Inverse Agonists

The following cyclic-AMP assay protocol using intact cells was used to determine inverse agonist activity.

Cells were plated into a 96-well plate at a plating density of 10,000-14,000 cells per well at a concentration of 100 μl per well. The plates were incubated for 24 hours in a 37° C. incubator. The media was removed and media lacking serum (100 μl) was added. The plates were then incubated for 18 hours at 37° C.

Serum free medium containing 1 mM IBMX was added to each well followed by 10 μl of test compound (1:10 stock solution (25 mM compound in DMSO) into 50% DMSO/PBS) diluted 10× in PBS with 0.1% BSA. After incubating for 20 minutes at 37° C., 2 μM of Forskolin was added and then incubated for an additional 20 minutes at 37° C. The media was removed, 100 μl of 0.01 N HCl was added and then incubated for 20 minutes at room temperature. Cell lysate (75 μl) along with 25 μl of assay buffer (supplied in FlashPlate™ cAMP assay kit available from NEN Life Science Products Boston, Mass.) into a Flashplate. cAMP standards and cAMP tracer were added following the kit's protocol. The flashplate was then incubated for 18 hours at 4° C. The content of the wells were aspirated and counted in a Scintillation counter.

### Alcohol Intake

The following protocol evaluates the effects of alcohol intake in alcohol preferring (P) female rats (bred at Indiana University) with an extensive drinking history. The following references provide detailed descriptions of P rats: Li, T. -K., et al., “Indiana selection studies on alcohol related behaviors” in Development of Animal Models as Pharmacogenetic Tools (eds McClearn C. E., Deitrich R. A. and Erwin V. G.), Research Monograph 6,171-192 (1981) NIAAA, ADAMHA, Rockville, Md.; Lumeng, L, et al., “New strains of rats with alcohol preference and nonpreference” Alcohol And Aldehyde Metabolizing Systems, 3, Academic Press, New York, 537-544 (1977); and Lumeng, L, et al., “Different sensitivities to ethanol in alcohol-preferring and -nonpreferring rats,” Pharmacol, Biochem Behav., 16, 125-130 (1982).

The female rats were given 2 hours of access to alcohol (10% v/v and water, 2-bottle choice) at the onset of the dark cycle. The rats were maintained on a reverse cycle to facilitate experimenter interactions. The rats were given subcutaneous water injections 3/1 and 3/4. The animals were assigned to four groups equated for intakes on March 4: Group 1—vehicle (n=8); Group 2—5.6 mg/kg AM251 (n=8); Group 3—10 mg/kg test compound (n=0); and Group 4—32 mg/kg test compound (n=8). Test compounds were mixed into a vehicle of 30% (w/v) β-cyclodextrin in distilled water. The AM251 would not form a solution in spite of extensive sonication and mixing; therefore, it was injected as a suspension while shaking the vessel prior to loading each syringe for accurate dosing. AM251 was injected at a volume of 2 ml/kg and the test compounds were injected at a volume of 1 ml/kg. On the drug injection days, drugs were given sc 30 minutes prior to a 2 hour alcohol access period. Drugs were injected on March 5 and March 6, 2001. No injections were given on March 7, but alcohol was available during the usual time. Alcohol intake for all animals was measured during the test period and a comparison was made between drug and vehicle-treated animals to determine effects of the compounds on alcohol drinking behavior.

### Hot Plate

Cannabinoid agonists have been shown to induce analgesia in male ICR mice; therefore, pre-treatment with a CB-1 antagonist should reverse the analgesia thereby providing a screen for in-vivo activity.

Male ICR mice (17-19 g) on arrival are pre-treated (n=8/treatment) with test compounds (sc, po, ip ot iv). Ten minutes later, mice were challenged with the CB agonist 5-(1,1-dimethyl-heptyl)-2-[5-hydroxy-2-(3-hydroxy-propyl)-cyclohexyl]-phenol (sc, ip, po or iv). Forty minutes later, each mouse was tested for reversal of analgesia using a standard hot plate meter (Columbus Instruments). The hot plate was 10″×10″×0.75″ with a surrounding clear acrylic wall. Latency to kick, lick or flick hindpaw or jump from the platform was recorded to the nearest tenth of a second. The timer was experimenter activated and each test had a 40 second cut off. Data was presented as a percent reversal of the agonist induced analgesia. The calculation used was (cp/agonist—veh/agonist)/(veh/veh—veh/agonist). Negative numbers indicated a potentiation of the agonist activity or non-antgonist activity; whereas, positive numbers indicated a reversal of the analgesia or antagonist activity.

What is claimed is:

1. A compound of Formula (I) 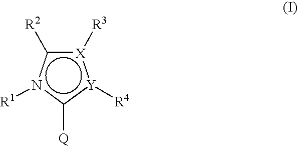
wherein
X is carbon and Y is nitrogen, or X is nitrogen and Y is carbon;
R¹ is a lone pair of electrons, hydrogen, (C₁-C₆)alkyl, or (C₃-C₆)cycloalkyl;
R² is hydrogen, (C₁-C₆)alkyl, or (C₃-C₆)cycloalkyl;
R³ is hydrogen or a chemical moiety selected from the group consisting of (C₁-C₆)alkyl, 2- to 8-membered carbocyclic ring, 5- to 6-membered heterocyclic ring, aryl, 5- to 9-membered heteroaryl, (C₁-C₆)alkylaryl, (C₁-C₆)alkylheteroaryl, and aryloxy(C₁-C₆)alkyl when X is carbon or nitrogen, where said chemical moiety is optionally substituted, or
R³ is a lone pair of electrons when X is nitrogen;
R⁴ is hydrogen or a chemical moiety selected from the group consisting of (C₁-C₆)alkyl, aryl, or aryl(C₁-C₆)alkyl when Y is carbon or nitrogen, where said chemical moiety is optionally substituted, or
R⁴ is a lone pair of electrons when Y is nitrogen; and
Q is a group selected from 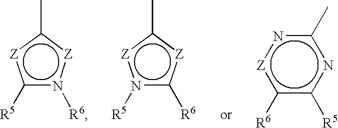
where Z in each occurrence is independently nitrogen or CR⁷, R⁵ is an optionally substituted aryl or an optionally substituted heteroaryl, R⁶is an optionally substituted aryl or an optionally substituted heteroaryl, and R⁷ is hydrogen, halo, cyano, or (C₁-C₆)alkyl;
a pharmaceutically acceptable salt thereof, a prodrug of said compound or said salt, or a solvate or hydrate of said compound, said salt or said prodrug.

2. The compound of claim 1 where R⁵ and R⁶ are each independently an aryl or a heteroaryl, where said aryl and said heteroaryl are substituted with one to three substituents selected from the group consisting of halo, (C₁-C₄)alkoxy, (C₁-C₄)alkyl, halo-substituted(C₁-C₄)alkyl and cyano.

3. The compound of claim 2 wherein R⁵ is 2,4-dihalophenyl or 2-halophenyl and R⁶ is 4-halophenyl or 2-(C₁-C₆)alkoxypyridin-5-yl;
a pharmaceutically acceptable salt thereof, a prodrug of said compound or said salt, or a solvate or hydrate of said compound, said salt or said prodrug.

4. The compound of claim 3 wherein R⁵ is 2,4-dichlorophenyl or 2-chlorophenyl and R⁶ is 4-chlorophenyl or 2-methoxypyridin-5-yl;
a pharmaceutically acceptable salt thereof, a prodrug of said compound or said salt, or a solvate or hydrate of said compound, said salt or said prodrug.

5. The compound of claim 1 selected from the group consisting of
5-(4-chloro-phenyl)-3-(5-cyclohexyl-1H-imidazol-2-yl)-1-(2,4-dichloro-phenyl)-4-methyl-1H-pyrazole;
5-(4-chloro-phenyl)-3-(2-cyclohexyl-3H-imidazol-4-yl )-1-(2,4-dichloro-phenyl)-4-methyl-1H-pyrazole;
5-(4-chloro-phenyl)-1-(2,4-dichloro-phenyl)-4-methyl-3-[1 -(1-methyl-1-phenyl-ethyl)-1H-imidazol-4-yl]-1H-pyrazole;
5-(4-chloro-phenyl)-1-(2-chloro-phenyl)-4-methyl-3-[1-(1-phenyl-ethyl)-1H-imidazol-4-yl]-1H-pyrazole;
5-(4-chloro-phenyl)-1-(2-fluoro-phenyl)-4-methyl-3-[1-(1-methyl-1-phenyl-ethyl)-1H-imidazol-4-yl]-1H-pyrazole;
5-(4-chloro-phenyl)-1-(2-chloro-phenyl)-3-[1-(2,2-dimethyl-tetrahydro-pyran-4-yl)-1H-imidazol-4-yl]-4-methyl-1H-pyrazole:
5-{2-(2,4-dichloro-phenyl)-4-methyl-5-[1 -(1-methyl-1-phenyl-ethyl)-1H-imidazol-4-yl]-2H-pyrazol-3-yl}-2-methoxy-pyridine; and
1-(2-chloro-phenyl)-5-(4-chloro-phenyl)-4-methyl-3-[1-(1-methyl-1-phenyl-ethyl)-1H-imidazol-4-yl]-1H-pyrazole;
a pharmaceutically acceptable salt thereof or a solvate or hydrate of said compound or said salt.

6. A compound having Formula (IA) or (IB) 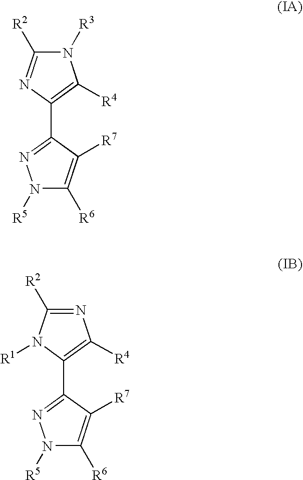
wherein
R¹ and R² are each independently hydrogen, (C₁-C₆)alkyl, or (C₃-C₆)cycloalkyl;
R³ is hydrogen or a chemical moiety selected from the group consisting of (C₁-C₆)alkyl, 2- to 8-membered carbocyclic ring, 5- to 6-membered heterocyclic ring, aryl, 5- to 9-membered heteroaryl, (C₁-C₆)alkylaryl, (C₁-C₆)alkylheteroaryl, and aryloxy(C₁-C₆)alkyl, where said chemical moiety is optionally substituted;
R⁴ is hydrogen or a chemical moiety selected from the group consisting of (C₁-C₆)alkyl, aryl, and aryl(C₁-C₆)alkyl, where said chemical moiety is optionally substituted;
R⁵ is an optionally substituted aryl or an optionally substituted heteroaryl;
R⁶ is an optionally substituted aryl or an optionally substituted heteroaryl; and
R⁷ is hydrogen, halo, cyano, or (C₁-C₆)alkyl;
a pharmaceutically acceptable salt thereof, a prodrug of said compound or said salt, or a solvate or hydrate of said compound, said salt or said prodrug.

7. The compound of claim 6 where R⁵ and R⁶ are each independently an aryl or a heteroaryl, where said aryl and said heteroaryl are substituted with one to three substituents selected from the group consisting of halo, (C₁-C₄)alkoxy, (C₁-C₄)alkyl, halo-substituted(C₁-C₄)alkyl and cyano.

8. The compound of claim 7 having Formula (IA); a pharmaceutically acceptable salt thereof, a prodrug of said compound or said salt, or a solvate or hydrate of said compound, said salt or said prodrug.

9. A compound having Formula (IC) or (ID) 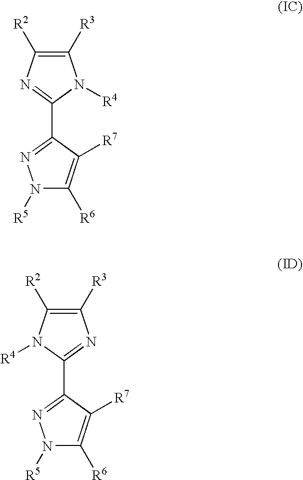
wherein
R¹ and R² are each independently hydrogen, (C₁-C₆)alkyl, or (C₃-C₆)cycloalkyl;
R³ is hydrogen or a chemical moiety selected from the group consisting of (C₁-C₆)alkyl, 2- to 8-membered carbocyclic ring, 5- to 6-membered heterocyclic ring, aryl, 5- to 9-membered heteroaryl, (C₁-C₆)alkylaryl, (C₁-C₆)alkylheteroaryl, and aryloxy(C₁-C₆)alkyl, where said chemical moiety is optionally substituted;
R⁴ is hydrogen or a chemical moiety selected from the group consisting of (C₁-C₆)alkyl, aryl, and aryl(C₁-C₆)alkyl, where said chemical moiety is optionally substituted;
R⁵ is an optionally substituted aryl, or an optionally substituted heteroaryl;
R⁶ is an optionally substituted aryl, or an optionally substituted heteroaryl; and
R⁷ is hydrogen, halo, cyano, or (C₁-C₆)alkyl;
a pharmaceutically acceptable salt thereof, a prodrug of said compound or said salt, or a solvate or hydrate of said compound, said salt or said prodrug.

10. The compound of claim 9 where R⁵ and R⁶ are each independently an aryl or a heteroaryl, where said aryl and said heteroaryl are substituted with one to three substituents selected from the group consisting of halo, (C₁-C₄)alkoxy, (C₁-C₄)alkyl, halo-substituted(C₁-C₄)alkyl and cyano.

11. The compound of claim 7, 8, or 10 wherein R⁵ is 2,4-dihalophenyl or 2-halophenyl and R⁶ is 4-halophenyl or 2-(C₁-C₆)alkoxypyrid in-5-yl;
a pharmaceutically acceptable salt thereof, a prodrug of said compound or said salt, or a solvate or hydrate of said compound, said salt or said prodrug.

12. The compound of claim 11 wherein R⁵ is 2,4-dichlorophenyl or 2-chlorophenyl and R⁶ is 4-chlorophenyl;
a pharmaceutically acceptable salt thereof, a prodrug of said compound or said salt, or a solvate or hydrate of said compound, said salt or said prodrug.

13. A pharmaceutical composition comprising (1) a compound of claim 1, a pharmaceutically acceptable salt thereof, a prodrug of said compound or said salt, or a solvate or hydrate of said compound, said salt or said prodrug; and (2) a pharmaceutically acceptable excipient, diluent, or carrier.

14. The pharmaceutical composition of claim 13 wherein said compound of claim 1 is a compound where R⁵ is 2,4-dichlorophenyl or 2-chlorophenyl and R⁶ is 4-chlorophenyl or 2-methoxypyridin-5-yl;
a pharmaceutically acceptable salt thereof, a prodrug of said compound or said salt, or a solvate or hydrate of said compound, said salt or said prodrug.

15. The pharmaceutical composition of claim 13 or 14 further comprising a nicotine partial agonist, an opioid antagonist, a dopaminergic agent, an attention deficit disorder agent, or an anti-obesity agent.

16. The composition of claim 15 wherein said anti-obesity agent is selected from the group consisting of an apo-B/MTP inhibitor, an 11β-hydroxy steroid dehydrogenase-1 inhibitor, peptide YY₃₋₃₆ or an analog thereof, a MCR-4 agonist, a CCK-A agonist, a monoamine reuptake inhibitor, a sympathomimetic agent, a β₃ adrenergic receptor agonist, a dopamine agonist, a melanocyte-stimulating hormone receptor analog, a 5-HT2c receptor agonist, a melanin concentrating hormone antagonist, leptin, a leptin analog, a leptin receptor agonist, a galanin antagonist, a lipase inhibitor, a bombesin agonist, a neuropeptide-Y receptor antagonist, a thyromimetic agent, dehydroepiandrosterone or analog thereof, a glucocorticoid receptor antagonist, an orexin receptor antagonist, a glucagon-like peptide-1 receptor agonist, a ciliary neurotrophic factor, a human agouti-related protein antagonist, a ghrelin receptor antagonist, a histamine 3 receptor antagonist or inverse agonist, and a neuromedin U receptor agonist.

17. A pharmaceutical composition comprising (1) a compound of claim 6, a pharmaceutically acceptable salt thereof, a prodrug of said compound or said salt, or a solvate or hydrate of said compound, said salt or said prodrug; and (2) a pharmaceutically acceptable excipient, diluent, or carrier.

18. The pharmaceutical composition of claim 17 wherein said compound of claim 6 is a compound where R⁵ is 2,4-dichlorophenyl or 2-chlorophenyl and R⁶ is 4-chlorophenyl or 2-methoxypyridin-5-yl;
a pharmaceutically acceptable salt thereof, a prodrug of said compound or said salt, or a solvate or hydrate of said compound, said salt or said prodrug.

19. The pharmaceutical composition of claim 17 or 18 further comprising a nicotine partial agonist, opioid antagonist, a dopaminergic agent, an attention deficit disorder agent, or an anti-obesity agent.

20. The composition of claim 19 wherein said anti-obesity agent is selected from the group consisting of an apo-B/MTP inhibitor, an 11β-hydroxy steroid dehydrogenase-1 inhibitor, peptide YY₃₋₃₆ or an analog thereof, a MCR-4 agonist, a CCK-A agonist, a monoamine reuptake inhibitor, a sympathomimetic agent, a β₃ adrenergic receptor agonist, a dopamine agonist, a melanocyte-stimulating hormone receptor analog, a 5-HT2c receptor agonist, a melanin concentrating hormone antagonist, leptin, a leptin analog, a leptin receptor agonist, a galanin antagonist, a lipase inhibitor, a bombesin agonist, a neuropeptide-Y receptor antagonist, a thyromimetic agent, dehydroepiandrosterone or analog thereof, a glucocorticoid receptor antagonist, an orexin receptor antagonist, a glucagon-like peptide-1 receptor agonist, a ciliary neurotrophic factor, a human agouti-related protein antagonist, a ghrelin receptor antagonist, a histamine 3 receptor antagonist or inverse agonist, and a neuromedin U receptor agonist.

21. A method for treating a disease, condition or disorder modulated by a cannabinoid receptor antagonist in animals comprising the step of administering to an animal in need of such treatment a therapeutically effective amount of a compound of claim 1, a pharmaceutically acceptable salt thereof, a prodrug of said compound or said salt, or a solvate or hydrate of said compound, said salt or said prodrug.

22. The method of claim 18 wherein said compound of claim 1 is a compound where R⁵ is 2,4-dichlorophenyl or 2-chlorophenyl and R⁶ is 4-chlorophenyl or 2-methoxypyridin-5-yl;
a pharmaceutically acceptable salt thereof, a prodrug of said compound or said salt, or a solvate or hydrate of said compound, said salt or said prodrug.

23. The method of claim 21 or 22 wherein said disease, condition or disorder modulated by a cannabinoid receptor antagonist is selected from the group consisting of eating disorders, weight loss or control, obesity, depression, atypical depression, bipolar disorders, psychoses, schizophrenia, behavioral addictions, suppression of reward-related behaviors, substance abuse, addictive disorders, impulsivity, alcoholism, tobacco abuse, dementia, sexual dysfunction in males, seizure disorders, epilepsy, gastrointestinal disorders, attention deficit disorder, Parkinson's disease, and type II diabetes.

24. The method of claim 23 wherein said disease, condition or disorder modulated by a cannabinoid receptor antagonist is obesity, alcoholism, attention deficit disorder, or tobacco abuse.

25. The method of claim 21 wherein said compound of claim 1 is administered in combination with a nicotine partial agonist, an opioid antagonist, a dopaminergic agent, an attention deficit disorder agent, or an anti-obesity agent.

26. The method of claim 25 wherein said anti-obesity agent is selected from the group consisting of an apo-B/MTP inhibitor, an IIβ-hydroxy steroid dehydrogenase-1 inhibitor, peptide YY₃₋₃₆ or an analog thereof, a MCR-4 agonist, a CCK-A agonist, a monoamine reuptake inhibitor, a sympathomimetic agent, a β₃ adrenergic receptor agonist, a dopamine agonist, a melanocyte-stimulating hormone receptor analog, a 5-HT2c receptor agonist, a melanin concentrating hormone antagonist, leptin, a leptin analog, a leptin receptor agonist, a galanin antagonist, a lipase inhibitor, a bombesin agonist, a neuropeptide-Y receptor antagonist, a thyromimetic agent, dehydroepiandrosterone or analog thereof, a glucocorticoid receptor antagonist, an orexin receptor antagonist, a glucagon-like peptide-1 receptor agonist, a ciliary neurotrophic factor, a human agouti-related protein antagonist, a ghrelin receptor antagonist, a histamine 3 receptor antagonist or inverse agonist, and a neuromedin U receptor agonist.

27. A method for treating a disease, condition or disorder modulated by a cannabinoid receptor antagonist in animals comprising the step of administering to an animal in need of such treatment a therapeutically effective amount of a compound of claim 6, a pharmaceutically acceptable salt thereof, a prodrug of said compound or said salt, or a solvate or hydrate of said compound, said salt or said prodrug.

28. The method of claim 27 wherein said compound of claim 6 is a compound where R⁵ is 2,4-dichlorophenyl or 2-chlorophenyl and R⁶ is 4-chlorophenyl or 2-methoxypyridin-5-yl;
a pharmaceutically acceptable salt thereof, a prodrug of said compound or said salt, or a solvate or hydrate of said compound, said salt or said prodrug.

29. The method of claim 27 or 28 said disease, condition or disorder modulated by a cannabinoid receptor antagonist is selected from the group consisting of eating disorders, weight loss or control, obesity, depression, atypical depression, bipolar disorders, psychoses, schizophrenia, behavioral addictions, suppression of reward-related behaviors, substance abuse, addictive disorders, impulsivity, alcoholism, tobacco abuse, dementia, sexual dysfunction in males, seizure disorders, epilepsy, gastrointestinal disorders, attention deficit disorder, Parkinson's disease, and type II diabetes.

30. The method of claim 29 wherein said disease, condition or disorder modulated by a cannabinoid receptor antagonist is obesity, alcoholism, attention deficit disorder, or tobacco abuse.

31. The method of claim 27 wherein said compound of claim 6 is administered in combination with a nicotine partial agonist, an opioid antagonist, a dopaminergic agent, an attention deficit disorder agent, or an anti-obesity agent.

32. The method of claim 31 wherein said anti-obesity agent is selected from the group consisting of an apo-B/MTP inhibitor, an 11β-hydroxy steroid dehydrogenase-1 inhibitor, peptide YY₃₋₃₆ or an analog thereof, a MCR-4 agonist, a CCK-A agonist, a monoamine reuptake inhibitor, a sympathomimetic agent, a 3 adrenergic receptor agonist, a dopamine agonist, a melanocyte-stimulating hormone receptor analog, a 5-HT2c receptor agonist, a melanin concentrating hormone antagonist, leptin, a leptin analog, a leptin receptor agonist, a galanin antagonist, a lipase inhibitor, a bombesin agonist, a neuropeptide-Y receptor antagonist, a thyromimetic agent, dehydroepiandrosterone or analog thereof, a glucocorticoid receptor antagonist, an orexin receptor antagonist, a glucagon-like peptide-1 receptor agonist, a ciliary neurotrophic factor, a human agouti-related protein antagonist, a ghrelin receptor antagonist, a histamine 3 receptor antagonist or inverse agonist, and a neuromedin U receptor agonist.
